SEQUENCE LISTING (1 sequence)

SEQ ID NO: 1 - protein, 1255 aa, Artificial Sequence [sequence omitted]

(19) United States
(12) Patent Application Publication
(10) Pub. No.: US 2023/0372342 A1
(43) Pub. Date: Nov. 23, 2023

(54) METHODS OF TREATING SOLID TUMORS DRIVEN BY HER2 ALTERATIONS WITH TUCATINIB IN COMBINATION WITH AN ANTI-HER2 ANTIBODY

(71) Applicant: Seagen Inc., Bothell, WA (US)

(72) Inventors: Luke WALKER, Bothell, WA (US); Anita KULUKIAN, Bothell, WA (US)

(21) Appl. No.: 18/027,557

(22) PCT Filed: Sep. 27, 2021
(86) PCT No.: PCT/US2021/071606

§ 371 Date: Mar. 21, 2023

Related U.S. Application Data
(60) Provisional application No. 63/222,335, filed on Jul. 15, 2021.
(60) Provisional application No. 63/113,245, filed on Nov. 13, 2020.
(60) Provisional application No. 63/084,481, filed on Sep. 28, 2020.

(51) Int. Cl. A61K 31/517; A61K 39/395; A61K 31/567

(52) U.S. Cl. CPC A61K 31/517; A61K 39/39558; A61K 31/567; A61K 2039/545

(57) ABSTRACT

The invention provides methods of treating solid tumors, such as solid tumors with HER2 alterations, e.g., solid tumors in which HER2 is amplified/overexpressed or mutated, with a combination of tucatinib, or salt or solvate thereof, and at least one anti-HER2 antibody, such as trastuzumab or trastuzumab and pertuzumab.

### CROSS-REFERENCE TO RELATED APPLICATIONS

This application claims the priority benefit of U.S. Provisional Application No. 63/084,481, filed on Sep. 28, 2020; U.S. Provisional Application No. 63/113,245, filed on Nov. 13, 2020, and U.S. Provisional Application No. 63/222,335, filed on Jul. 15, 2021, the disclosures of which are each incorporated herein by reference for all purposes.

### TECHNICAL FIELD

The present invention relates to methods of treating solid tumors, such as solid tumors with HER2 alterations, e.g., solid tumors in which HER2 is amplified/overexpressed or mutated, with a combination of tucatinib, or salt or solvate thereof, and at least one anti-HER2 antibody, such as trastuzumab.

### BACKGROUND

Encoded by the ERBB2 gene, human epidermal growth factor receptor 2 (HER2) is part of a family of 4 related receptor tyrosine kinases, which include HER1 (also known as epidermal growth factor receptor [EGFR]), HER2, HER3, and HER4. HER1-4 are single-pass transmembrane glycoprotein receptors containing an extracellular ligand binding region and an intracellular signaling domain. HER2 has no known ligand, but it is the preferred dimerization partner for the other HER family receptors. When overexpressed in tumors, HER2 forms ligand-independent homodimeric complexes that autophosphorylate. HER2 homo- or heterodimerization results in the activation of multiple signaling cascades, including the Ras/Raf/MEK/MAPK, PI3K/AKT, Src, and STAT pathways. These signaling pathways lead to cell proliferation, inhibition of apoptosis, and metastasis.

HER2 is a validated target in multiple solid tumors, with anti-HER2 biologics and small molecule-drugs approved for patients with HER2 overexpressing/amplified breast and gastric cancers. Amplification of the HER2 gene or overexpression of its protein occurs in approximately 15% to 20% of breast cancers.

In typical HER2+ cancers, including breast cancer, gastric cancer, and colorectal cancer, the amplification of HER2 leads to strong signal transduction through either homodimerization or heterodimerization with another ErbB-family member. This results in downstream activation of both the MAP kinase and phosphatidyl-inositol-3 (PI3) kinase pathways, which in turn enhances mitogenicity and survival.

In some cancers, however, HER2 expression is not amplified, but rather HER2 may contain an activating mutation in the kinase domain that also leads to increased signaling and mitogenicity. See WO 2018/200505. HER2 activating mutations may act as oncogenic drivers in various cancer types. See WO 2018/200505. The majority of these HER2-mutant cancers have not been associated with concurrent HER2 gene amplification, with the result that an important subgroup of HER2-altered cancers are not detected by immunohistochemistry (IHC) or in situ hybridization (ISH) methods. In the clinic, they can be identified by next generation sequencing (NGS) in either tumor biopsies or circulating cell-free DNA (cfDNA). Annals of Oncol 28:136-141 (2017). Preclinical data indicate that HER2 “hot spot” mutations may be constitutively active, have transforming capacity in vitro and in vivo and may show variable sensitivity to anti-HER2 based therapies. J Mol Diagn, 17(5):487-495 (2015), Nat Gen 51, 207-216 (2019). Recent clinical trials also revealed potential activity of HER2-targeted drugs against a variety of tumors harboring HER2 mutations. HER2-targeted agents could potentially be useful for the treatment of cancers harboring these activating mutations. ESMO Open 2017; 2: e000279. However, efforts to target cancers with HER2 mutations have met with limited clinical success, possibly because of their low frequency, inadequate understanding of the biological activity of these mutations, and difficulty in separating the drivers from the passenger mutations. The Oncologist 24(12):e1303-e1314 (2019). The role of HER2-directed therapy in these HER2-mutated cancers is the subject of active exploration.

Tucatinib ((N′-(4-([1,2,4]triazolo[1,5-a]pyridin-7-yloxy)-3-methylphenyl)-N⁶-(4,4-dimethyl-4,5-dihydrooxazol-2-yl) quinazoline-4,6-diamine) (TUKYSA™; formerly known as ARRY-380 and ONT-380) is an orally (PO) administered, potent, highly selective, small-molecule tyrosine kinase inhibitor (TKI) of HER2. Tucatinib is a potent inhibitor of HER2 in vitro, and in cellular signaling assays is >1000-fold more selective for HER2 compared to the closely related kinase EGFR. The selectivity of tucatinib for HER2 reduces the potential for EGFR-related toxicities that can be seen with dual HER2/EGFR inhibitors. Tucatinib inhibits the HER2-driven mitogen-activated protein and PI3 kinase signaling pathways, resulting in inhibition of tumor cell proliferation, survival, and metastasis.

Tucatinib, combined with trastuzumab and capecitabine, is approved for use in previously treated patients with advanced unresectable or metastatic HER2+ breast cancer in Australia, Canada, Singapore, Switzerland, and the US.

Trastuzumab, a humanized anti-HER2 antibody that binds to the HER2 extracellular domain, is approved for use in the treatment of HER2+ breast cancer and remains the backbone of treatment in the perioperative and metastatic setting, usually in combination with a taxane. Pertuzumab is another approved anti-HER2 monoclonal antibody, which binds to the HER2 receptor at a site different from trastuzumab.

All references cited herein, including patent applications, patent publications, and scientific literature, are herein incorporated by reference in their entirety, as if each individual reference were specifically and individually indicated to be incorporated by reference.

### SUMMARY

Provided herein is a method for treating a solid tumor in a subject comprising administering a combination of tucatinib, or salt or solvate thereof, and at least one anti-HER2 antibody to the subject, wherein the solid tumor has one or more HER2 alterations. In some embodiments, the subject exhibits progression free survival (PFS) of at least 1 month after administration of the tucatinib, or salt or solvate thereof, and the at least one anti-HER2 antibody. In some embodiments, the subject exhibits an overall survival (OS) of at least 2 months after administration of the tucatinib, or salt or solvate thereof, and the at least one anti-HER2 antibody. In some embodiments, the subject exhibits a greater than 10% reduction in the risk of disease progression as compared to a subject administered the at least one anti-HER2 antibody alone. In some embodiments, the subject exhibits a greater than 25% reduction in the risk of disease progression as compared to a subject administered the at least one anti-HER2 antibody alone. In some embodiments, the subject exhibits a greater than 30% reduction in the risk of disease progression as compared to a subject administered the at least one anti-HER2 antibody alone. In some embodiments, the subject exhibits a greater than 10% reduction in the risk of death as compared to a subject administered the at least one anti-HER2 antibody alone. In some embodiments, the subject exhibits a greater than 25% reduction in the risk of death as compared to a subject administered the at least one anti-HER2 antibody alone. In some embodiments, the subject exhibits a greater than 30% reduction in the risk of death as compared to a subject administered the at least one anti-HER2 antibody alone. In some embodiments, following administration of the tucatinib, or salt or solvate thereof, and the at least one anti-HER2 antibody, for nine months the subject has an estimated PFS rate of greater than 20%. In some embodiments, the subject has an estimated PFS rate of greater than 30%. In some embodiments, the subject has an estimated PFS rate of greater than 40%. In some embodiments, the subject has an estimated PFS rate of greater than 45%. In some embodiments, following administration of the tucatinib, or salt or solvate thereof, and the at least one anti-HER2 antibody, for twelve months the subject has an estimated PFS rate of greater than 15%. In some embodiments, the subject has an estimated PFS rate of greater than 20%. In some embodiments, the subject has an estimated PFS rate of greater than 30%. In some embodiments, the subject has an estimated PFS rate of greater than 40%. In some embodiments, following administration of the tucatinib, or salt or solvate thereof, and the at least one anti-HER2 antibody, for fifteen months the subject has an estimated PFS rate of greater than 15%. In some embodiments, the subject has an estimated PFS rate of greater than 20%. In some embodiments, the subject has an estimated PFS rate of greater than 25%. In some embodiments, the subject has an estimated PFS rate of greater than 30%. In some embodiments, following administration of the tucatinib, or salt or solvate thereof, and the at least one anti-HER2 antibody, for twenty-four months the subject has an estimated OS rate of greater than 25%. In some embodiments, the subject has an estimated OS rate of greater than 35%. In some embodiments, the subject has an estimated OS rate of greater than 40%. In some embodiments, following administration of the tucatinib, or salt or solvate thereof, and the at least one anti-HER2 antibody, for thirty months the subject has an estimated OS rate of greater than 20%. In some embodiments, the subject has an estimated OS rate of greater than 25%. In some embodiments, the subject has an estimated OS rate of greater than 30%. In some embodiments, the tucatinib, or salt or solvate thereof, and at least one anti-HER2 antibody are administered to the subject on a 21-day treatment cycle. In some embodiments, the at least one anti-HER2 antibody is administered to the subject on day 1 of the 21-day treatment cycle. In some embodiments, the at least one anti-HER2 antibody is administered once about every 3 weeks. In some embodiments, the tucatinib, or salt or solvate thereof, is administered to the subject twice per day. In some embodiments, the one or more HER2 alterations is a HER2 mutation, wherein the HER2 mutation comprises at least one amino acid substitution, insertion, or deletion compared to the amino acid sequence of SEQ ID NO:1. In some embodiments, the HER2 mutation is an activating mutation. In some embodiments, the HER2 mutation is a mutation in the extracellular domain, the kinase domain, or the transmembrane/juxtamembrane domain, or any combination thereof. In some embodiments, the HER2 mutation is a mutation in the extracellular domain selected from the group consisting of G309A, G309E, S310F, S310Y, C311R, C311S, and C334S. In some embodiments, the HER2 mutation is a mutation in the kinase domain at an amino acid residue selected from the group consisting of Y772, G776, G778, and T798. In some embodiments, the HER2 mutation is a G776 YVMA insertion. In some embodiments, the HER2 mutation is a mutation in the kinase domain selected from the group consisting of T733I, L755P, L755S, I767M, L768S, D769N, D769Y, D769H, V777L, V777M, L841V, V8421, N857S, T862A, L869R, H878Y, and R896C. In some embodiments, the HER2 mutation is a mutation in the kinase domain at an amino acid residue V697. In some embodiments, the HER2 mutation is a mutation in the transmembrane/juxtamembrane domain selected from the group consisting of S653C, 1655V, V659E, G660D, and R678Q. In some embodiments, the HER2 mutation is determined by using next generation sequencing (NGS). In some embodiments, the one or more HER2 alterations is HER2 overexpression/amplification. In some embodiments, the HER2 overexpression is 3+ overexpression as determined by immunohistochemistry (IHC). In some embodiments, the HER2 amplification is determined by an in situ hybridization assay. In some embodiments, the in situ hybridization assay is fluorescence in situ hybridization (FISH) assay. In some embodiments, the in situ hybridization assay is chromogenic in situ hybridization. In some embodiments, the HER2 amplification is determined in tissue by NGS. In some embodiments, the HER2 amplification is determined in circulating tumor DNA (ctDNA) by a blood-based NGS assay. In some embodiments, the solid tumor is a HER2+ solid tumor. In some embodiments, the solid tumor is a metastatic solid tumor. In some embodiments, the solid tumor is locally-advanced. In some embodiments, the solid tumor is unresetable. In some embodiments, the solid tumor is selected from the group consisting of cervical cancer, uterine cancer, gallbladder cancer, cholangiocarcinoma, urothelial cancer, lung cancer, breast cancer, gastroesophageal cancer, and colorectal cancer. In some embodiments, the solid tumor is gallbladder cancer and the subject has completed at least one prior line of treatment for the gallbladder cancer. In some embodiments, the prior line of treatment is selected from the group consisting of chemotherapy, endocrine therapy, and targeted therapy. In some embodiments, the solid tumor is cholangiocarcinoma and the subject has completed at least one prior line of treatment for the cholangiocarcinoma. In some embodiments, the prior line of treatment is selected from the group consisting of chemotherapy, endocrine therapy, and targeted therapy. In some embodiments, the lung cancer is non-small cell lung cancer (NSCLC). In some embodiments, the NSCLC is non-squamous NSCLC. In some embodiments, the subject has relapsed from standard of care treatment. In some embodiments, the subject is refractory to standard of care treatment. In some embodiments, no standard of care treatment is available for the subject. In some embodiments, the solid tumor is breast cancer. In some embodiments, the subject has completed at least one prior line of treatment for the breast cancer. In some embodiments, the prior line of treatment is selected from the group consisting of chemotherapy, endocrine therapy, and targeted therapy. In some embodiments, the breast cancer is hormone receptor positive (HR+) breast cancer. In some embodiments, the breast cancer is a metastasized breast cancer. In some embodiments, the subject is administered fulvestrant in combination with the tucatinib, or salt or solvate thereof, and the at least one anti-HER2 antibody. In some embodiments, the fluvestrant is administered at a dose of 500 mg. In some embodiments, the route of administration of the fluvestrant is administered intramuscular (IM). In some embodiments, the fluvestrant is administered on day 1 of the first 21-day treatment cycle. In some embodiments, the fluvestrant is administered once about every 4 weeks. In some embodiments, the fluvestrant is further administered on day 15 of the first 21-day treatment cycle. In some embodiments, the tucatinib, or salt or solvate thereof, is administered to the subject at a dose of about 150 mg to about 650 mg. In some embodiments, the tucatinib, or salt or solvate thereof, is administered to the subject at a dose of about 300 mg. In some embodiments, the tucatinib, or salt or solvate thereof, is administered to the subject orally. In some embodiments, the at least one anti-HER2 antibody is administered to the subject at a dose of about 4 mg/kg to about 10 mg/kg. In some embodiments, the at least one anti-HER2 antibody is administered to the subject at a dose of about 6 mg/kg of the subject's body weight. In some embodiments, the at least one anti-HER2 antibody is administered to the subject at a dose of about 8 mg/kg of the subject's body weight. In some embodiments, the at least one anti-HER2 antibody is administered to the subject at a dose of about an initial dose of about 8 mg/kg followed by subsequent doses of about 6 mg/kg. In some embodiments, the dose of the at least one anti-HER2 antibody administered during the first 21-day treatment cycle is 8 mg/kg of the subject's body weight and the dose administered during the subsequent 21-day treatment cycles is 6 mg/kg of the subject's bodyweight. In some embodiments, the at least one anti-HER2 antibody is administered intravenously. In some embodiments, the at least one anti-HER2 antibody comprises one anti-HER2 antibody. In some embodiments, the at least one anti-HER2 antibody is no more than one anti-HER2 antibody. In some embodiments, the at least one anti-HER2 antibody is trastuzumab, or a biosimilar thereof. In some embodiments, the at least one anti-HER2 antibody is trastuzumab. In some embodiments, the at least one anti-HER2 antibody comprises a first anti-HER2 antibody and a second anti-HER2 antibody. In some embodiments, the first anti-HER2 antibody is administered to the subject at a dose of about 4 mg/kg to about 10 mg/kg. In some embodiments, the first anti-HER2 antibody is administered to the subject at a dose of about 6 mg/kg of the subject's body weight. In some embodiments, the first anti-HER2 antibody is administered at a dose of about 200 mg to about 1,000 mg. In some embodiments, the first anti-HER2 antibody is administered at a dose of about 600 mg. In some embodiments, the second anti-HER2 antibody is administered to the subject at a dose of about 200 mg to about 1.000 mg. In some embodiments, the second anti-HER2 antibody is administered at a dose of about 420 mg. In some embodiments, the second anti-HER2 antibody is administered at a dose of about 600 mg. In some embodiments, the first anti-HER2 antibody is administered at a dose of about 6 mg/kg and the second anti-HER2 antibody is administered at a dose of about 420 mg. In some embodiments, the first anti-HER2 antibody is administered intravenously. In some embodiments, the first anti-HER2 antibody is administered subcutaneously. In some embodiments, the second anti-HER2 antibody is administered intravenously. In some embodiments, the second anti-HER2 antibody is administered subcutaneously. In some embodiments, the first anti-HER2 antibody is administered at a dose of 6 mg/kg intravenously or at a dose of about 600 mg subcutaneously; and wherein the second anti-HER2 antibody is administered at a dose of about 420 mg intravenously. In some embodiments, the first anti-HER2 antibody and the second anti-HER2 antibody are administered in a pharmaceutical composition comprising about 600 mg of the first anti-HER2 antibody and 600 mg of the second anti-HER2 antibody; wherein the pharmaceutical composition is administered subcutaneously. In some embodiments, the pharmaceutical composition further comprises hyaluronidase. In some embodiments, the pharmaceutical composition comprises about 20,000 units hyaluronidase. In some embodiments, the first anti-HER2 antibody is trastuzumab, or a biosimilar thereof. In some embodiments, the second anti-HER2 antibody is pertuzumab, or a biosimilar thereof. In some embodiments, the first anti-HER2 antibody is administered about once every 3 weeks. In some embodiments, the second anti-HER2 antibody is administered about once every 3 weeks. In some embodiments, treating the subject results in a tumor growth inhibition (TGI) index of at least about 85%. In some embodiments, treating the subject results in a TGI index of about 100%. In some embodiments, one or more therapeutic effects in the subject is improved after administration of the tucatinib, or salt or solvate thereof, and the at least one anti-HER2 antibody to the subject relative to a baseline. In some embodiments, the one or more therapeutic effects is selected from the group consisting of: size of a tumor derived from the solid tumor, objective response rate, duration of response, time to response, progression free survival and overall survival. In some embodiments, the size of a tumor derived from the solid tumor is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the solid tumor before administration of the tucatinib, or salt or solvate thereof, and the at least one anti-HER2 antibody. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody. In some embodiments, the subject exhibits overall survival of at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody. In some embodiments, the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody. In some embodiments, the administration of the tucatinib, or salt or solvate thereof, increases the overall amount of HER2 in the solid tumor. In some embodiments, the overall amount of HER2 in the solid tumor is determined by western blot analysis. In some embodiments, the administration of the tucatinib, or salt or solvate thereof, increases the amount of plasma membrane-bound HER2 in the solid tumor. In some embodiments, the amount of plasma membrane-bound HER2 in the solid tumor is determined by quantitative fluorescence activated cell sorting (qFACS). In some embodiments, the administration of the tucatinib, or salt or solvate thereof, increases the dwell time of HER2 at the cell surface. In some embodiments, the administration of the tucatinib, or salt or solvate thereof, increases the internalization of plasma membrane-bound HER2. In some embodiments, the administration of the tucatinib, or salt or solvate thereof, increases the lysosomal degradation of HER2. In some embodiments, the subject is a human.

Also provided herein is a method of increasing the overall amount of HER2 in a solid tumor comprising administering tucatinib, or salt or solvate thereof to a subject, wherein the administration of the tucatinib, or salt or solvate thereof, increases the overall amount of HER2 in the solid tumor. In some embodiments, the overall amount of HER2 in the solid tumor is determined by Western blot analysis.

Also provided herein is a method of increasing the amount of plasma membrane-bound HER2 in a solid tumor comprising administering tucatinib, or salt or solvate thereof to a subject, wherein the administration of the tucatinib, or salt or solvate thereof, increases the amount of plasma membrane-bound HER2 in the solid tumor. In some embodiments, the amount of plasma membrane-bound HER2 in the solid tumor is determined by quantitative fluorescence activated cell sorting (qFACS).

Also provided herein is a method of increasing dwell time of HER2 at the cell surface of a solid tumor comprising administering tucatinib, or salt or solvate thereof to a subject, wherein the administration of the tucatinib, or salt or solvate thereof, increases the dwell time of HER2 at the cell surface.

Also provided herein is a method of increasing internalization of plasma membrane-bound HER2 in a solid tumor comprising administering tucatinib, or salt or solvate thereof to a subject, wherein the administration of the tucatinib, or salt or solvate thereof, increases the internalization of plasma membrane-bound HER2.

Also provided herein is a method of increasing lysosomal degradation of HER2 in a solid tumor comprising administering tucatinib, or salt or solvate thereof to a subject, wherein the administration of the tucatinib, or salt or solvate thereof, increases the lysosomal degradation of HER2.

Also provided herein is a kit comprising tucatinib, or salt or solvate thereof, at least one anti-HER2 antibody, and instructions for using the kit according to any of the embodiments herein. In some embodiments, the at least one anti-HER2 antibody comprises trastuzumab. In some embodiments, the at least one anti-HER2 antibody comprises pertuzumab.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1H are a series of graphs showing that treatment with tucatinib increased overall and plasma membrane-bound HER2 levels in BT-474, SK-BR-3, HCC-1419, and UACC-893 cell lines after treatment with tucatinib at either 30 nM or 100 nM doses for the duration of 24 hours and 48 hours.

FIGS. 2A-2D are a series of schematics and images showing that treatment with tucatinib increased dwell time of HER2 at the cell surface and was followed by rapid internalization and lysosomal processing. FIG. 2A shows a schematic of a HER2 internalization assay using Trastuzumab-AF488. FIG. 2B shows the results of a HER2 internalization assay using Trastuzumab-AF488. FIG. 2C shows a schematic of a HER2 internalization assay using Trastuzumab-QF. FIG. 2D shows the results of a HER2 internalization assay using Trastuzumab-QF (small box in corner shows staining of cells using DAPI).

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C: A or C; A or B; B or C; A and C; A and B: B and C; A (alone); B (alone); and C (alone).

It is understood that aspects and embodiments of the invention described herein include “comprising,” “consisting,” and “consisting essentially of” aspects and embodiments.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B: A, B, and C; A, B, C. and D; A, B, C, D, and E; and the like.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of an anti-HER2 antibody and tucatinib. In some instances, the co-administered compounds are administered by the same route. In other instances, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that an anti-HER2 antibody and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “T0” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression. Mol Biol Int. 2014:852748 (2014). The scoring method may be based on the cell membrane staining pattern as determined by immunohistochemistry and is as follows:
i. 3+: positive HER2 expression, uniform intense membrane staining of more than 30% of invasive tumor cells;
ii. 2+: equivocal for HER2 protein expression, complete membrane staining that is either nonuniform or weak in intensity but has circumferential distribution in at least 10% of cells;
iii. 0 or 1+: negative for HER2 protein expression.

The term “tucatinib,” also known as ONT-380 and ARRY-380, refers to the small molecule tyrosine kinase inhibitor that suppresses or blocks HER2 activation. Tucatinib has the following structure:

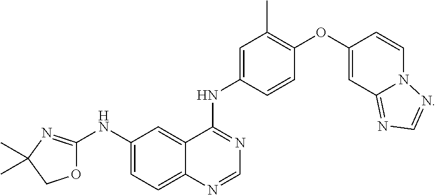 [SMILES: Cc1cc(Nc2ncnc3ccc(NC4=NC(C)(C)CO4)cc23)ccc1Oc1ccn2ncnc2c1]

The term “anti-HER2 antibody” refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, ado-trastuzumab emtansine (also known as T-DM1), margetuximab, and combinations thereof.

The term “chemotherapeutic agent” refers to a group of compounds useful in treating or ameliorating cancer or its symptoms. In some embodiments, chemotherapeutic agents include alkylating antineoplastic agents (e.g., nitrogen mustards, such as mechlorathamine, isfosfamide, melphalan, chlorambucil, and cyclophosphamide: alkyl sufonates, such as busulfan: nitrosoureas, such as streptozocin, carmustine, and lomustine: triazines, such as dacarbazine and temozolonide; and ethyleneimines, such as thiotepa and altretamine), antimetabolites (see below), antitumor anmibiotics (e.g., the anthracycins, such as daunorubicin, doxorubicin, epirubicin, idarubicin, and valrubicin: the bleomycins: Mitomycin C, mitoxantrone, and actinomycin), aromatase inhibitors (e.g., steroidal inhibitors, such as exemestane: and non-steroidal inhibitors, such as anastrozole and letrozole), kinase inhibitors (e.g., tyrosine kinase inhibitors, such as imatinib, gefitinib, erlotinib, lapatinib, nilotinib, sunitibnib, and sorafenib; and, e.g., bosunitinib, neratinib, vatalanib, and toceranib), mTor inhibitors (e.g., rapamycin and its analogs, such as tensirolimus, everolimus, and ridaforolimus; dual PIcK/mTOR inhibitors; and ATP-competitive mTOR inhibitors, such as sapanisertib), retinoids (e.g., tretinoin, alitretinoin, bexarotene, and isotretinoin), topoisomerase inhibitors (e.g., doxorubicin, etoposide, teniposide, mitoxantrone, novobiocin, merbaron, aclatubicin, camptothecin, and camptothecin prodrugs or derivatives, such as irinotecan and topothecan), and plant alkaloids (e.g., the Vinca alkaloids vinblastine, vinorelbine, vincristine, and vindesine; the taxanes, such as docetaxel and paclitaxel).

The term “tumor growth inhibition (TGI) index” refers to a value used to represent the degree to which an agent (e.g., tucatinib described herein, an anti-HER2 antibody described herein, or a combination thereof) inhibits the growth of a tumor when compared to an untreated control. The TGI index is calculated for a particular time point (e.g., a specific number of days into an experiment or clinical trial) according to the following formula:

$$T G I = 1 - ( \frac{{Volume}_{treated ( Tx Day X )} - {Volume}_{treated ( Tx Day 0 )}}{{Volume}_{control ( Tx Day X )} - {Volume}_{control ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib and at least one anti-HER2 antibody) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and assumes that the drugs act completely independently (i.e., the drugs do not interfere with one another (e.g., the drugs have different sites of action) but each contributes to a common result). According to the Bliss Independence model, the predicted effect of a combination of two drugs is calculated using the formula:

E_{AB}=E_{A}+E_{B}−E_{A}×E_{B}.

where E_{A} and E_{B} represent the effects of drugs A and B, respectively, and E_{AB} represents the effect of a combination of drugs A and B. When the observed effect of the combination is greater than the predicted effect E_{AB}, then the combination of the two drugs is considered to be synergistic. When the observed effect of the combination is equal to E_{AB}, then the effect of the combination of the two drugs is considered to be additive. Alternatively, when the observed effect of the combination is less than E_{AB}, then the combination of the two drugs is considered to be antagonistic.

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

The predicted combination effect E_{AB} can be calculated using either a single dose or multiple doses of the drugs that make up the combination (e.g., tucatinib and at least one anti-HER2 antibody). In some embodiments, the predicted combination effect E_{A}B is calculated using only a single dose of each drug A and B (e.g., tucatinib and at least one anti-HER2 antibody), and the values E_{A} and E_{B} are based on the observed effect of each drug when administered as a single agent. When the values for E_{A} and E_{B} are based on the observed effects of administering drugs A and B as single agents, E_{A} and E_{B} can be based on, for example, TGI indices, tumor sizes (e.g., volume, mass) measured at one or more time points, absolute changes in tumor size (e.g., volume, mass) between two or more time points (e.g., between the first day a treatment is adminstered and a particular number of days after treatment is first administered), the rates of change of tumor sizes (e.g., volume, mass) between two or more time points (e.g., between the first day a treatment is adminstered and a particular number of days after treatment is first administered), or the survival time of a subject or a population of subjects in each treatment group.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, the predicted combination effect E_{AB} is calculated using a range of doses (i.e., the effects of each drug, when administered as a single agent, are observed at multiple doses and the observed effects at the multiple doses are used to determine the predicted combination effect at a specific dose). As a non-limiting example, E_{AB} can be calculated using values for E_{A} and E_{B} that are calculated according to the following formulae:

$${E}_{A} = {E}_{Amax} \times \frac{{a}^{p}}{{A}_{50}^{p} + {a}^{p}}$$

$${E}_{B} = {E}_{Bmax} \times \frac{{b}^{q}}{{B}_{50}^{q} + {b}^{q}} ,$$

where E_{Amax} and E_{Bmax} are the maximum effects of drugs A and B, respectively, A₅₀ and B₅₀ are the half maximum effective doses of drugs A and B, respectively, a and b are administered doses of drugs A and B, respectively, and p and q are coefficients that are derived from the shapes of the dose-response curves for drugs A and B, respectively (see, e.g., Foucquier et al. Pharmacol. Res. Perspect. (2015) 3(3):e00149).

In some embodiments, a combination of two or more drugs is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug (e.g., tucatinib) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD: and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 6.0 mg/kg of an agent, such as trastuzumab, one can calculate and use the appropriate amount of the agent (i.e., 360 mg) for administration to said subject.

The use of the term “fixed dose” with regard to a method of the disclosure means that two or more different agents (e.g., tucatinb and anti-HER2 antibody) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 1:2 ratio of tucatinib to an anti-HER2 antibody administered to a subject can mean about 300 mg of tucatinib and about 600 mg of the at least one anti-HER2 antibody or about 3 mg/ml of tucatinib and about 6 mg/ml of the at least one anti-HER2 antibody are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib or anti-HER2 antibody). For example, a subject with 60 kg body weight and a subject with 100 kg body weight would receive the same dose of tucatinb (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present invention.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value: a mean value: or a value as compared to a reference value. The reference value and/or baseline value can be obtained from one individual, from two different individuals or from a group of individuals (e.g., a group of two, three, four, five or more individuals).

The term “monotherapy” as used herein means that the tucatinib, or salt or solvate thereof, is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating a Solid Tumor with Tucatinib and at Least One Anti-HER2 Antibody

In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein the subject exhibits progression free survival (PFS) of at least 1 month after administration of the tucatinib, or salt or solvate thereof, and the at least one anti-HER2 antibody. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein the subject exhibits progression free survival (PFS) of at least 1 month after administration of the tucatinib, or salt or solvate thereof, and the at least one anti-HER2 antibody. In some embodiments, the subject exhibits PFS of at least 2 months after administration of the tucatinib, or salt or solvate thereof, and the at least one anti-HER2 antibody. In some embodiments, the subject exhibits PFS of at least 3 months after administration of the tucatinib, or salt or solvate thereof, and the at least one anti-HER2 antibody. In some embodiments, the subject exhibits PFS of at least 4 months after administration of the tucatinib, or salt or solvate thereof, and the at least one anti-HER2 antibody. In some embodiments, the subject exhibits PFS of at least 5 months after administration of the tucatinib, or salt or solvate thereof, and the at least one anti-HER2 antibody. In some embodiments, the subject exhibits PFS of at least 6 months after administration of the tucatinib, or salt or solvate thereof, and the at least one anti-HER2 antibody. In some embodiments, the subject exhibits PFS of at least 7 months after administration of the tucatinib, or salt or solvate thereof, and the at least one anti-HER2 antibody. In some embodiments, the subject exhibits PFS of at least 8 months after administration of the tucatinib, or salt or solvate thereof, and the at least one anti-HER2 antibody. In some embodiments, the subject exhibits PFS of at least 9 months after administration of the tucatinib, or salt or solvate thereof, and the at least one anti-HER2 antibody. In some embodiments, the subject exhibits PFS of at least 10 months after administration of the tucatinib, or salt or solvate thereof, and the at least one anti-HER2 antibody. In some embodiments, the subject exhibits PFS of at least 11 months after administration of the tucatinib, or salt or solvate thereof, and the at least one anti-HER2 antibody. In some embodiments, the subject exhibits PFS of at least 12 months after administration of the tucatinib, or salt or solvate thereof, and the at least one anti-HER2 antibody. In some embodiments, the subject exhibits PFS of at least 15 months after administration of the tucatinib, or salt or solvate thereof, and the at least one anti-HER2 antibody. In some embodiments, the subject exhibits PFS of at least 18 months after administration of the tucatinib, or salt or solvate thereof, and the at least one anti-HER2 antibody. In some embodiments, the subject exhibits PFS of at least 2 years after administration of the tucatinib, or salt or solvate thereof, and the at least one anti-HER2 antibody. In some embodiments, the subject exhibits PFS of at least 3 years after administration of the tucatinib, or salt or solvate thereof, and the at least one anti-HER2 antibody. In some embodiments, the subject exhibits PFS of at least 4 years after administration of the tucatinib, or salt or solvate thereof, and the at least one anti-HER2 antibody. In some embodiments, the subject exhibits PFS of at least 5 years after administration of the tucatinib, or salt or solvate thereof, and the at least one anti-HER2 antibody. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein the subject exhibits an overall survival (OS) of at least 2 months after administration of the tucatinib, or salt or solvate thereof, and the at least one anti-HER2 antibody. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein the subject exhibits an overall survival (OS) of at least 2 months after administration of the tucatinib, or salt or solvate thereof, and the at least one anti-HER2 antibody. In some embodiments, the subject exhibits OS of at least 3 months after administration of the tucatinib, or salt or solvate thereof, and the at least one anti-HER2 antibody. In some embodiments, the subject exhibits OS of at least 4 months after administration of the tucatinib, or salt or solvate thereof, and the at least one anti-HER2 antibody. In some embodiments, the subject exhibits OS of at least 5 months after administration of the tucatinib, or salt or solvate thereof, and the at least one anti-HER2 antibody. In some embodiments, the subject exhibits OS of at least 6 months after administration of the tucatinib, or salt or solvate thereof, and the at least one anti-HER2 antibody. In some embodiments, the subject exhibits OS of at least 7 months after administration of the tucatinib, or salt or solvate thereof, and the at least one anti-HER2 antibody. In some embodiments, the subject exhibits OS of at least 8 months after administration of the tucatinib, or salt or solvate thereof, and the at least one anti-HER2 antibody. In some embodiments, the subject exhibits OS of at least 9 months after administration of the tucatinib, or salt or solvate thereof, and the at least one anti-HER2 antibody. In some embodiments, the subject exhibits OS of at least 10 months after administration of the tucatinib, or salt or solvate thereof, and the at least one anti-HER2 antibody. In some embodiments, the subject exhibits OS of at least 11 months after administration of the tucatinib, or salt or solvate thereof, and the at least one anti-HER2 antibody. In some embodiments, the subject exhibits OS of at least 12 months after administration of the tucatinib, or salt or solvate thereof, and the at least one anti-HER2 antibody. In some embodiments, the subject exhibits OS of at least 15 months after administration of the tucatinib, or salt or solvate thereof, and the at least one anti-HER2 antibody. In some embodiments, the subject exhibits OS of at least 18 months after administration of the tucatinib, or salt or solvate thereof, and the at least one anti-HER2 antibody. In some embodiments, the subject exhibits OS of at least 2 years after administration of the tucatinib, or salt or solvate thereof, and the at least one anti-HER2 antibody. In some embodiments, the subject exhibits OS of at least 3 years after administration of the tucatinib, or salt or solvate thereof, and the at least one anti-HER2 antibody. In some embodiments, the subject exhibits OS of at least 4 years after administration of the tucatinib, or salt or solvate thereof, and the at least one anti-HER2 antibody. In some embodiments, the subject exhibits OS of at least 5 years after administration of the tucatinib, or salt or solvate thereof, and the at least one anti-HER2 antibody. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein the subject exhibits a greater than 10% reduction in the risk of disease progression as compared to a subject administered the at least one anti-HER2 antibody alone. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein the subject exhibits a greater than 10% reduction in the risk of disease progression as compared to a subject administered the at least one anti-HER2 antibody alone. In some embodiments, the subject exhibits a greater than 15% reduction in the risk of disease progression as compared to a subject administered the at least one anti-HER2 antibody alone. In some embodiments, the subject exhibits a greater than 20% reduction in the risk of disease progression as compared to a subject administered the at least one anti-HER2 antibody alone. In some embodiments, the subject exhibits a greater than 25% reduction in the risk of disease progression as compared to a subject administered the at least one anti-HER2 antibody alone. In some embodiments, the subject exhibits a greater than 30% reduction in the risk of disease progression as compared to a subject administered the at least one anti-HER2 antibody alone. In some embodiments, the subject exhibits a greater than 35% reduction in the risk of disease progression as compared to a subject administered the at least one anti-HER2 antibody alone. In some embodiments, the subject exhibits a greater than 40% reduction in the risk of disease progression as compared to a subject administered the at least one anti-HER2 antibody alone. In some embodiments, the subject exhibits a greater than 45% reduction in the risk of disease progression as compared to a subject administered the at least one anti-HER2 antibody alone. In some embodiments, the subject exhibits a greater than 55% reduction in the risk of disease progression as compared to a subject administered the at least one anti-HER2 antibody alone. In some embodiments, the subject exhibits a greater than 60% reduction in the risk of disease progression as compared to a subject administered the at least one anti-HER2 antibody alone. In some embodiments, the subject exhibits a greater than 65% reduction in the risk of disease progression as compared to a subject administered the at least one anti-HER2 antibody alone. In some embodiments, the subject exhibits a greater than 70% reduction in the risk of disease progression as compared to a subject administered the at least one anti-HER2 antibody alone. In some embodiments, the subject exhibits a greater than 75% reduction in the risk of disease progression as compared to a subject administered the at least one anti-HER2 antibody alone. In some embodiments, the subject exhibits a greater than 80% reduction in the risk of disease progression as compared to a subject administered the at least one anti-HER2 antibody alone. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein the subject exhibits a greater than 10% reduction in the risk of death as compared to a subject administered the at least one anti-HER2 antibody alone. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein the subject exhibits a greater than 10% reduction in the risk of death as compared to a subject administered the at least one anti-HER2 antibody alone. In some embodiments, the subject exhibits a greater than 15% reduction in the risk of death as compared to a subject administered the at least one anti-HER2 antibody alone. In some embodiments, the subject exhibits a greater than 20% reduction in the risk of death as compared to a subject administered the at least one anti-HER2 antibody alone. In some embodiments, the subject exhibits a greater than 25% reduction in the risk of death as compared to a subject administered the at least one anti-HER2 antibody alone. In some embodiments, the subject exhibits a greater than 30% reduction in the risk of death as compared to a subject administered the at least one anti-HER2 antibody alone. In some embodiments, the subject exhibits a greater than 35% reduction in the risk of death as compared to a subject administered the at least one anti-HER2 antibody alone. In some embodiments, the subject exhibits a greater than 40% reduction in the risk of death as compared to a subject administered the at least one anti-HER2 antibody alone. In some embodiments, the subject exhibits a greater than 45% reduction in the risk of death as compared to a subject administered the at least one anti-HER2 antibody alone. In some embodiments, the subject exhibits a greater than 50% reduction in the risk of death as compared to a subject administered the at least one anti-HER2 antibody alone. In some embodiments, the subject exhibits a greater than 55% reduction in the risk of death as compared to a subject administered the at least one anti-HER2 antibody alone. In some embodiments, the subject exhibits a greater than 60% reduction in the risk of death as compared to a subject administered the at least one anti-HER2 antibody alone. In some embodiments, the subject exhibits a greater than 65% reduction in the risk of death as compared to a subject administered the at least one anti-HER2 antibody alone. In some embodiments, the subject exhibits a greater than 70% reduction in the risk of death as compared to a subject administered the at least one anti-HER2 antibody alone. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein following administration of the tucatinib, or salt or solvate thereof, and the at least one anti-HER2 antibody, for nine months the subject has an estimated PFS rate of greater than 20%. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein following administration of the tucatinib, or salt or solvate thereof, and the at least one anti-HER2 antibody, for nine months the subject has an estimated PFS rate of greater than 20%. In some embodiments, the subject has an estimated PFS rate of greater than 25%. In some embodiments, the subject has an estimated PFS rate of greater than 30%. In some embodiments, the subject has an estimated PFS rate of greater than 35%. In some embodiments, the subject has an estimated PFS rate of greater than 40%. In some embodiments, the subject has an estimated PFS rate of greater than 45%. In some embodiments, the subject has an estimated PFS rate of greater than 50%. In some embodiments, the subject has an estimated PFS rate of greater than 55%. In some embodiments, the subject has an estimated PFS rate of greater than 60%. In some embodiments, the subject has an estimated PFS rate of greater than 65%. In some embodiments, the subject has an estimated PFS rate of greater than 70%. In some embodiments, the subject has an estimated PFS rate of greater than 75%. In some embodiments, the subject has an estimated PFS rate of greater than 80%. In some embodiments, the subject has an estimated PFS rate of greater than 85%. In some embodiments, the subject has an estimated PFS rate of greater than 85%. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein following administration of the tucatinib, or salt or solvate thereof, and the at least one anti-HER2 antibody, for twelve months the subject has an estimated PFS rate of greater than 15%. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein following administration of the tucatinib, or salt or solvate thereof, and the at least one anti-HER2 antibody, for twelve months the subject has an estimated PFS rate of greater than 15%. In some embodiments, the subject has an estimated PFS rate of greater than 20%. In some embodiments, the subject has an estimated PFS rate of greater than 25%. In some embodiments, the subject has an estimated PFS rate of greater than 30%. In some embodiments, the subject has an estimated PFS rate of greater than 35%. In some embodiments, the subject has an estimated PFS rate of greater than 40%. In some embodiments, the subject has an estimated PFS rate of greater than 45%. In some embodiments, the subject has an estimated PFS rate of greater than 50%. In some embodiments, the subject has an estimated PFS rate of greater than 55%. In some embodiments, the subject has an estimated PFS rate of greater than 60%. In some embodiments, the subject has an estimated PFS rate of greater than 65%. In some embodiments, the subject has an estimated PFS rate of greater than 70%. In some embodiments, the subject has an estimated PFS rate of greater than 75%. In some embodiments, the subject has an estimated PFS rate of greater than 80%. In some embodiments, the subject has an estimated PFS rate of greater than 85%. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein following administration of the tucatinib, or salt or solvate thereof, and the at least one anti-HER2 antibody, for fifteen months the subject has an estimated PFS rate of greater than 15%. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein following administration of the tucatinib, or salt or solvate thereof, and the at least one anti-HER2 antibody, for fifteen months the subject has an estimated PFS rate of greater than 15%. In some embodiments, the subject has an estimated PFS rate of greater than 20%. In some embodiments, the subject has an estimated PFS rate of greater than 25%. In some embodiments, the subject has an estimated PFS rate of greater than 30%. In some embodiments, the subject has an estimated PFS rate of greater than 35%. In some embodiments, the subject has an estimated PFS rate of greater than 40%. In some embodiments, the subject has an estimated PFS rate of greater than 45%. In some embodiments, the subject has an estimated PFS rate of greater than 50%. In some embodiments, the subject has an estimated PFS rate of greater than 55%. In some embodiments, the subject has an estimated PFS rate of greater than 60%. In some embodiments, the subject has an estimated PFS rate of greater than 65%. In some embodiments, the subject has an estimated PFS rate of greater than 70%. In some embodiments, the subject has an estimated PFS rate of greater than 75%. In some embodiments, the subject has an estimated PFS rate of greater than 80%. In some embodiments, the subject has an estimated PFS rate of greater than 85%. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein following administration of the tucatinib, or salt or solvate thereof, and the at least one anti-HER2 antibody, for twenty-four months the subject has an estimated OS rate of greater than 25%. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein following administration of the tucatinib, or salt or solvate thereof, and the at least one anti-HER2 antibody, for twenty-four months the subject has an estimated OS rate of greater than 25%. In some embodiments, the subject has an estimated OS rate of greater than 30%. In some embodiments, the subject has an estimated OS rate of greater than 35%. In some embodiments, the subject has an estimated OS rate of greater than 40%. In some embodiments, the subject has an estimated OS rate of greater than 45%. In some embodiments, the subject has an estimated OS rate of greater than 50%. In some embodiments, the subject has an estimated OS rate of greater than 55%. In some embodiments, the subject has an estimated OS rate of greater than 60%. In some embodiments, the subject has an estimated OS rate of greater than 65%. In some embodiments, the subject has an estimated OS rate of greater than 70%. In some embodiments, the subject has an estimated OS rate of greater than 75%. In some embodiments, the subject has an estimated OS rate of greater than 80%. In one aspect, the present invention provides a method for treating a solid tumor in a subject comprising administering a combination of tucatinib, or salt or solvate thereof, and at least one anti-HER2 antibody to the subject, wherein the solid tumor comprises a HER2 alteration, wherein following administration of the tucatinib, or salt or solvate thereof, and the at least one anti-HER2 antibody, for thirty months the subject has an estimated OS rate of greater than 20%. In one aspect, the present invention provides a method for treating a solid tumor in a subject comprising administering a combination of tucatinib, or salt or solvate thereof, and at least one anti-HER2 antibody to the subject, wherein the solid tumor has one or more HER2 alterations, wherein following administration of the tucatinib, or salt or solvate thereof, and the at least one anti-HER2 antibody, for thirty months the subject has an estimated OS rate of greater than 20%. In some embodiments, the subject has an estimated OS rate of greater than 25%. In some embodiments, the subject has an estimated OS rate of greater than 30%. In some embodiments, the subject has an estimated OS rate of greater than 35%. In some embodiments, the subject has an estimated OS rate of greater than 40%. In some embodiments, the subject has an estimated OS rate of greater than 45%. In some embodiments, the subject has an estimated OS rate of greater than 50%. In some embodiments, the subject has an estimated OS rate of greater than 55%. In some embodiments, the subject has an estimated OS rate of greater than 60%. In some embodiments, the subject has an estimated OS rate of greater than 65%. In some embodiments, the subject has an estimated OS rate of greater than 70%. In some embodiments, the subject has an estimated OS rate of greater than 75%. In some embodiments, the subject has an estimated OS rate of greater than 80%. In some embodiments, the administration of the tucatinib, or salt or solvate thereof, increases the overall amount of HER2 in the solid tumor. In one aspect, the invention provides a method of increasing the overall amount of HER2 in a solid tumor comprising administering tucatinib, or salt or solvate thereof to a subject, wherein the administration of the tucatinib, or salt or solvate thereof, increases the overall amount of HER2 in the solid tumor. In some embodiments, the amount of HER2 in the solid tumor is determined by Western blot analysis. In some embodiments, the amount of HER2 in the solid tumor is determined by immunohistochemistry. In some embodiments, the amount of HER2 in the solid tumor is determined by mass spectrometry. In some embodiments, the amount of HER2 in the solid tumor is determined by ELISA. In some embodiments, the amount of HER2 in the solid tumor is determined by real-time quantitative PCR (qRT-PCR). In some embodiments, the amount of HER2 in the solid tumor is determined by microarray analysis. In some embodiments, the administration of the tucatinib, or salt or solvate thereof, increases the amount of plasma membrane-bound HER2 in the solid tumor. In one aspect, the invention provides a method of increasing the amount of plasma membrane-bound HER2 in a solid tumor comprising administering tucatinib, or salt or solvate thereof to a subject, wherein the administration of the tucatinib, or salt or solvate thereof, increases the amount of plasma membrane-bound HER2 in the solid tumor. In some embodiments, the amount of plasma membrane-bound HER2 in the solid tumor is determined by quantitative fluorescence activated cell sorting (qFACS). In some embodiments, the administration of the tucatinib, or salt or solvate thereof, increases the dwell time of HER2 at the cell surface. In one aspect, the invention provides a method of increasing dwell time of HER2 at the cell surface of a solid tumor comprising administering tucatinib, or salt or solvate thereof to a subject, wherein the administration of the tucatinib, or salt or solvate thereof, increases the dwell time of HER2 at the cell surface. In some embodiments, the administration of the tucatinib, or salt or solvate thereof, increases the internalization of plasma membrane-bound HER2. In one aspect, the invention provides a method of increasing internalization of plasma membrane-bound HER2 in a solid tumor comprising administering tucatinib, or salt or solvate thereof to a subject, wherein the administration of the tucatinib, or salt or solvate thereof, increases the internalization of plasma membrane-bound HER2. In some embodiments, the administration of the tucatinib, or salt or solvate thereof, increases the lysosomal degradation of HER2. In one aspect, the invention provides a method of increasing lysosomal degradation of HER2 in a solid tumor comprising administering tucatinib, or salt or solvate thereof to a subject, wherein the administration of the tucatinib, or salt or solvate thereof, increases the lysosomal degradation of HER2. In some embodiments, the subject is a human.

In some embodiments, the one or more HER2 alterations is a HER2 mutation. In some at least one amino acid substitution, insertion, or deletion compared to the human wild-type HER2 amino acid sequence. In some embodiments, human wild-type HER2 comprises the amino acid sequence of

(SEQ ID NO: 1)
MELAALCRWGLLLALLPPGAASTQVCTGTDMKLRLPASPET
HLDMLRHLYQGCQVVQGNLELTYLPTNASLSFLQDIQEVQ
GYVLIAHNQVRQVPLQRLRIVRGTQLFEDNYALAVLDNGD
PLNNTTPVTGASPGGLRELQLESLTEILKGGVLIQRNPQL
CYQDTILWKDIFHKNNQLALTLIDTNRSRACHPCSPMCKG
SRCWGESSEDCQSLTRTVCAGGCARCKGPLPTDCCHEQCA
AGCTGPKHSDCLACLHFNHSGICELHCPALVTYNTDTFES
MPNPEGRYTFGASCVTACPYNYLSTDVGSCTLVCPLHNQE
VTAEDGTQRCEKCSKPCARVCYGLGMEHLREVRAVTSANI
QEFAGCKKIFGSLAFLPESFDGDPASNTAPLQPEQLQVFE
TLEEITGYLYISAWPDSLPDLSVFQNLQVIRGRILHNGAY
SLTLQGLGISWLGLRSLRELGSGLALIHHNTHLCFVHTVP
WDQLFRNPHQALLHTANRPEDECVGEGLACHQLCARGHCW
GPGPTQCVNCSQFLRGQECVEECRVLQGLPREYVNARHCL
PCHPECQPQNGSVTCFGPEADQCVACAHYKDPPFCVARCP
SGVKPDLSYMPIWKFPDEEGACQPCPINCTHSCVDLDDKG
CPAEQRASPLTSIISAVVGILLVVVLGVVFGILIKRRQQK
IRKYTMRRLLQETELVEPLTPSGAMPNQAQMRILKETELR
KVKVLGSGAFGTVYKGIWIPDGENVKIPVAIKVLRENTSP
KANKEILDEAYVMAGVGSPYVSRLLGICLTSTVQLVTQLM
PYGCLLDHVRENRGRLGSQDLLNWCMQIAKGMSYLEDVRL
VHRDLAARNVLVKSPNHVKITDFGLARLLDIDETEYHADG
GKVPIKWMALESILRRRFTHQSDVWSYGVTVWELMTFGAK
PYDGIPAREIPDLLEKGERLPQPPICTIDVYMIMVKCWMI
DSECRPRFRELVSEFSRMARDPQRFVVIQNEDLGPASPLD
STFYRSLLEDDDMGDLVDAEEYLVPQQGFFCPDPAPGAGG
MVHHRHRSSSTRSGGGDLTLGLEPSEEEAPRSPLAPSEGA
GSDVFDGDLGMGAAKGLQSLPTHDPSPLQRYSEDPTVPLP
SETDGYVAPLTCSPQPEYVNQPDVRPQPPSPREGPLPAAR
PAGATLERPKTLSPGKNGVVKDVFAFGGAVENPEYLTPQG
GAAPQPHPPPAFSPAFDNLYYWDQDPPERGAPPSTFKGTP
TAENPEYLGLDVPV.

In some embodiments, the HER2 mutation is an activating mutation. In some embodiments, the HER2 mutation results in constitutive HER2 kinase domain activation. In some embodiments, the HER2 mutation is a mutation in the extracellular domain, the kinase domain, or the transmembrane/juxtamembrane domain, or any combination thereof. In some embodiments, the HER2 mutation is a mutation in the extracellular domain. In some embodiments, the HER2 mutation is a mutation in the extracellular domain selected from the group consisting of G309A, G309E, S310F, S310Y, C311R, C311S, and C334S. In some embodiments, the mutation in the extracellular domain is G309A. In some embodiments, the mutation in the extracellular domain is G309E. In some embodiments, the mutation in the extracellular domain is S310F. In some embodiments, the mutation in the extracellular domain is S310Y. In some embodiments, the mutation in the extracellular domain is C311R. In some embodiments, the mutation in the extracellular domain is C311S. In some embodiments, the mutation in the extracellular domain is C334S. In some embodiments, the HER2 mutation is a mutation in the kinase domain. In some embodiments, the HER2 mutation is a mutation in the kinase domain at an amino acid residue selected from the group consisting of Y772, G776, G778, and T798. In some embodiments, the mutation in the kinase domain is at Y772. In some embodiments, the mutation in the kinase domain is at G776. In some embodiments, the mutation at G776 is a G776 YVMA insertion (G776 ins YVMA). The G776 ins YVMA mutant form of HER2 is a mutant in which YVMA (tyrosine, valine, methionine, alanine), which is the amino acid sequence at positions 772 to 775 of the HER2 protein, is repeated once again (also referred to as “Y772_A775dup” or “A775_G776insYVMA”). Nature. 2004 Sep. 30; 431 (7008): 525-6, and Cancer Res. 2005 Mar. 1; 65 (5): 1642-6. In some embodiments, the mutation in the kinase domain is at G778. In some embodiments, the mutation in the kinase domain is at T798. In some embodiments, the HER2 mutation is a mutation in the kinase domain selected from the group consisting of T733I, L755P, L755S, I767M, L768S, D769N, D769Y, D769H, V777L, V777M, L841V, V8421, N857S, T862A, L869R, H878Y, and R896C. In some embodiments, the mutation in the kinase domain is T733I. In some embodiments, the mutation in the kinase domain is L755P. In some embodiments, the mutation in the kinase domain is L755S. In some embodiments, the mutation in the kinase domain is I767M. In some embodiments, the mutation in the kinase domain is L768S. In some embodiments, the mutation in the kinase domain is D769N. In some embodiments, the mutation in the kinase domain is D769Y. In some embodiments, the mutation in the kinase domain is D769H. In some embodiments, the mutation in the kinase domain is V777L. In some embodiments, the mutation in the kinase domain is V777M. In some embodiments, the mutation in the kinase domain is L841V. In some embodiments, the mutation in the kinase domain is V8421. In some embodiments, the mutation in the kinase domain is N857S. In some embodiments, the mutation in the kinase domain is T862A. In some embodiments, the mutation in the kinase domain is L869R. In some embodiments, the mutation in the kinase domain is H878Y. In some embodiments, the mutation in the kinase domain is R896C. In some embodiments, the HER2 mutation is a mutation in the transmembrane/juxtamembrane domain. In some embodiments, the HER2 mutation is a mutation in the kinase domain at an amino acid residue V697. In some embodiments, the HER2 mutation is a mutation in the transmembrane/juxtamembrane domain selected from the group consisting of S653C, I655V, V659E, G660D, and R678Q. In some embodiments, the mutation in the transmembrane/juxtamembrane domain is S653C. In some embodiments, the mutation in the transmembrane/juxtamembrane domain is I655V. In some embodiments, the mutation in the transmembrane/juxtamembrane domain is V659E. In some embodiments, the mutation in the transmembrane/juxtamembrane domain is G660D. In some embodiments, the mutation in the transmembrane/juxtamembrane domain is R678Q. In some embodiments, the cancer does not have HER2 amplification. In some embodiments, the cancer has been determined to not comprise a HER2 amplification. In some embodiments, HER2 amplification is determined by IHC. In some embodiments, the cancer has a HER2 amplification score of 0, wherein the HER2 amplification score is determined by IHC. In some embodiments, the cancer has a HER2 amplification score of 1+, wherein the HER2 amplification score is determined by IHC. In some embodiments, the cancer has a HER2 amplification score of 0 or 1+, wherein the HER2 amplification score is determined by IHC. In some embodiments, HER2 is not amplified if the cancer has a score of 0 as determined by IHC. In some embodiments, HER2 is not amplified if the cancer has a score of 1+ as determined by IHC. In some embodiments, the HER2 mutation is determined by DNA sequencing. In some embodiments, the HER2 mutation is determined by RNA sequencing. In some embodiments, the HER2 mutation is determined by using next generation sequencing (NGS). In some embodiments, the HER2 mutation is determined by polymerase chain reaction (PCR).

In some embodiments, the one or more HER2 alterations is a HER2 overexpression/amplification. In some embodiments, the cancer has a HER2 amplification score of 2+, wherein the HER2 amplification score is determined by immunohistochemistry (IHC). In some embodiments, the cancer has a HER2 amplification score of 3+, wherein the HER2 amplification score is determined by IHC. In some embodiments, HER2 is amplified if the cancer has a score of 2+ as determined by IHC. In some embodiments, HER2 is amplified if the cancer has a score of 3+ as determined by IHC. In some embodiments, HER2 is amplified if it is overexpressed in the cancer by at least about 5%, about 10%, about 15%, about 20%, about 25%, about 30%, about 35%, about 40%, about 45%, about 50%, about 55%, about 60%, about 70%, about 75%, about 80%, about 85%, about 90%, about 95%, about 100%, about 125%, about 150%, about 175%, about 200%, about 250%, about 300%, about 350%, about 400%, about 450%, or about 500%. In some embodiments, HER2 is amplified if it is overexpressed in the cancer by at least 50%. In some embodiments, HER2 is amplified if it is overexpressed in the cancer by at least 75%. In some embodiments, HER2 is amplified if it is overexpressed in the cancer by at least 100%. In some embodiments, HER2 is amplified if it is overexpressed in the cancer by at least 150%. In some embodiments, HER2 is amplified if it is overexpressed in the cancer by at least 200%. In some embodiments, HER2 is amplified if it is overexpressed in the cancer by at least 250%. In some embodiments, HER2 is amplified if it is overexpressed in the cancer by at least 300%. In some embodiments, HER2 is amplified if it is overexpressed in the cancer by at least 400%. In some embodiments, HER2 is amplified if it is overexpressed in the cancer by at least 500%. In some embodiments, HER2 is amplified if there is at least about a 1.5 fold, about a 2 fold, about a 3 fold, about a 4 fold, about a 5 fold, about a 10 fold, about a 15 fold, about a 20 fold, about a 25 fold, about a 30 fold, about a 40 fold, about a 50 fold, about a 60 fold, about a 70 fold, about a 80 fold, about a 90 fold, or about a 100 fold increase in HER2 protein levels in the cancer. In some embodiments, HER2 is amplified if there is at least about a 1.5 fold increase in HER2 protein levels in the cancer. In some embodiments, HER2 is amplified if there is at least about a 2 fold increase in HER2 protein levels in the cancer. In some embodiments, HER2 is amplified if there is at least about a 3 fold increase in HER2 protein levels in the cancer. In some embodiments, HER2 is amplified if there is at least about a 4 fold increase in HER2 protein levels in the cancer. In some embodiments, HER2 is amplified if there is at least about a 5 fold increase in HER2 protein levels in the cancer. In some embodiments, HER2 is amplified if there is at least about a 10 fold increase in HER2 protein levels in the cancer. In some embodiments, HER2 is amplified if there is at least about a 15 fold increase in HER2 protein levels in the cancer. In some embodiments, HER2 is amplified if there is at least about a 20 fold increase in HER2 protein levels in the cancer. In some embodiments. HER2 is amplified if there is at least about a 25 fold increase in HER2 protein levels in the cancer. In some embodiments, HER2 is amplified if there is at least about a 30 fold increase in HER2 protein levels in the cancer. In some embodiments. HER2 is amplified if there is at least about a 40 fold increase in HER2 protein levels in the cancer. In some embodiments, HER2 is amplified if there is at least about a 50 fold increase in HER2 protein levels. In some embodiments, HER2 is amplified if there is at least about a 60 fold increase in HER2 protein levels in the cancer. In some embodiments. HER2 is amplified if there is at least about a 70 fold increase in HER2 protein levels in the cancer. In some embodiments, HER2 is amplified if there is at least about an 80 fold increase in HER2 protein levels in the cancer. In some embodiments, HER2 is amplified if there is at least about a 90 fold increase in HER2 protein levels in the cancer. In some embodiments, HER2 is amplified if there is at least about a 100 fold increase in HER2 protein levels in the cancer. In some embodiments, the HER2 overexpression is 3+ overexpression as determined by immunohistochemistry (IHC). In some embodiments, the HER2 amplification is determined by an in situ hybridization assay. In some embodiments, the in situ hybridization assay is fluorescence in situ hybridization (FISH) assay. In some embodiments, the in situ hybridization assay is chromogenic in situ hybridization. In some embodiments, the HER2 amplification is determined in tissue by NGS. In some embodiments, the HER2 amplification is determined in circulating tumor DNA (ctDNA) by a blood-based NGS assay.

In some embodiments, the solid tumor is a HER2+ solid tumor. In some embodiments, the solid tumor is a metastatic solid tumor. In some embodiments, the solid tumor is locally-advanced. In some embodiments, the solid tumor is unresetable. In some embodiments, the subject has been previously treated with one or more additional therapeutic agents for the solid tumor. In some embodiments, the subject has been previously treated with one or more additional therapeutic agents for the solid tumor and did not respond to the treatment. In some embodiments, the subject has been previously treated with one or more additional therapeutic agents for the solid tumor and relapsed after the treatment. In some embodiments, the subject has been previously treated with one or more additional therapeutic agents for the solid tumor and experienced disease progression during the treatment. In some embodiments, the solid tumor is selected from the group consisting of cervical cancer, uterine cancer, gallbladder cancer, cholangiocarcinoma, urothelial cancer, lung cancer, breast cancer, gastroesophageal cancer, and colorectal cancer. In some embodiments, the solid tumor is cervical cancer. In some embodiments, the solid tumor is uterine cancer. In some embodiments, the solid tumor is a biliary tract cancer, e.g., gallbladder cancer or cholangiocarcinoma. In some embodiments, the solid tumor is a biliary tract cancer and the subject has completed at least one prior line of treatment for the gallbladder cancer. In some embodiments, the prior line of treatment is selected from the group consisting of chemotherapy, endocrine therapy, and targeted therapy. In some embodiments, the solid tumor is gallbladder cancer. In some embodiments, the solid tumor is gallbladder cancer and the subject has completed at least one prior line of treatment for the gallbladder cancer. In some embodiments, the prior line of treatment is selected from the group consisting of chemotherapy, endocrine therapy, and targeted therapy. In some embodiments, the solid tumor is cholangiocarcinoma. In some embodiments, the solid tumor is cholangiocarcinoma and the subject has completed at least one prior line of treatment for the cholangiocarcinoma. In some embodiments, the prior line of treatment is selected from the group consisting of chemotherapy, endocrine therapy, and targeted therapy. In some embodiments, the solid tumor is urothelial cancer. In some embodiments, the solid tumor is lung cancer. In some embodiments, the lung cancer is non-small cell lung cancer (NSCLC). In some embodiments, the NSCLC is non-squamous NSCLC. In some embodiments, the NSCLC is non-squamous NSCLC and the subject has relapsed from standard of care treatment. In some embodiments, the NSCLC is non-squamous NSCLC and the subject is refractory to standard of care treatment. In some embodiments, the NSCLC is non-squamous NSCLC and no standard of care treatment is available for the subject. In some embodiments, the solid tumor is breast cancer. In some embodiments, the subject has completed at least one prior line of treatment for the breast cancer. In some embodiments, the prior line of treatment is selected from the group consisting of chemotherapy, endocrine therapy, and targeted therapy. In some embodiments, the breast cancer is hormone receptor positive (HR+) breast cancer. In some embodiments, the solid tumor is gastroesophageal cancer. In some embodiments, the solid tumor is colorectal cancer.

In some embodiments, the HER2 status of a sample cell is determined. The determination can be made before treatment (i.e., administration of tucatinib) begins, during treatment, or after treatment has been completed. In some instances, determination of the HER2 status results in a decision to change therapy (e.g., adding an anti-HER2 antibody to the treatment regimen, discontinuing the use of tucatinib, discontinuing therapy altogether, or switching from another treatment method to a method of the present invention).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA or RNA sequencing), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the presence or absence of a HER2 mutation is confirmed by, for example, collecting tumor tissue from a cancer patient and performing a method such as real-time quantitative PCR (qRT-PCR) or microarray analysis. In some embodiments, the tumor tissue is a formalin-fixed paraffin-embedded specimen (FFPE). In some embodiments, the presence or absence of HER2 mutation is confirmed by collecting acellular circulating tumor DNA (ctDNA) from a cancer patient and performing a method such as next generation sequencing (NGS) (J Clin Oncol 2013: 31: 1997-2003, Clin Cancer Res 2012; 18: 4910-8, J Thorac Oncol 2012: 7: 85-9, Lung Cancer 2011; 74: 139-44, Cancer Res 2005: 65: 1642-6, Cancer Sci 2006; 97: 753-9, and ESMO Open 2017; 2: e000279).

Nucleic acids used to detect HER2 mutations in any of the methods described herein include genomic DNA, RNA transcribed from genomic DNA, and cDNA generated from RNA. Nucleic acids can be derived from vertebrates, for example mammals. A nucleic acid is said to be directly derived from a particular source or “derived from” a particular source if it is a copy of a nucleic acid found in that source.

In certain embodiments, the nucleic acid comprises a copy of the nucleic acid, e.g., a copy resulting from amplification. For example, amplification to obtain the desired amount of material to detect mutations may be desirable in certain instances. The amplicon may then go through a mutation detection method, such as those described below, to determine whether the mutation is present in the amplicon.

Somatic mutations or variations can be detected by certain methods known to those skilled in the art. Such methods include, but are not limited to, DNA sequencing, primers including somatic mutation-specific nucleotide incorporation assays and somatic mutation-specific primer extension assays (e.g., somatic mutation-specific PCR, somatic mutation-specific ligation chain reaction (LCR), and gap-LCR extension assays), mutation-specific oligonucleotide hybridization assays (e.g., oligonucleotide ligation assays), cleavage protection assays in which protection from cleavage agents is used to detect fluorinated bases in nucleic acid duplexes, electrophoretic analysis comparing the mobility of variants and wild type nucleic acid molecules, denaturation-gradient gel electrophoresis (e.g., DGGE as in Myers et al. (1985) Nature 313: 495), analysis of RNase cleavage on uninched base pairs, analysis of chemical or enzymatic cleavage of heteroduplex DNA, mass spectrometry (e.g., MALDI-TOF); genetic bit analysis (GBA), 5 ′nuclease assay (e.g., TaqMan™), and assays using molecular pathway labels.

Detection of variation in the target nucleic acid can be accomplished by molecular cloning and sequencing of the target nucleic acid using techniques well known in the art. Alternatively, amplification techniques such as polymerase chain reaction (PCR) can be used to amplify target nucleic acid sequences directly from genomic DNA preparations from tumor tissue. The nucleic acid sequence of the amplified sequence can then be determined and variations identified therefrom. Amplification techniques are well known in the art, for example, polymerase chain reactions are described in Saiki et al., Science 239: 487, 1988; U.S. Pat. Nos. 4,683,203 and 4,683,195.

Ligase chain reactions known in the art can also be used to amplify target nucleic acid sequences. See, e.g., Wu et al., Genomics 4: 560-569 (1989). Also, a technique known as allele-specific PCR can also be used to detect somatic mutations (e.g., substitutions). See, e.g., Ruano and Kidd (1989) Nucleic Acids Research 17: 8392; McClay et al. (2002) Analytical Biochem. 301: 200-206. In certain embodiments of this technique, the 3 ′terminal nucleotides of the primers are complementary to (i.e., specifically form base pairs with) certain variations of the target nucleic acid. Mutation-specific primers are used. If no specific mutation is present, no amplification product is observed. Amplification resistance mutation systems (ARMS) can also be used to detect variations (e.g., substitutions). ARMS is described, for example, in European Patent Application Publication No. 0332435, and Newton et al., Nucleic Acids Research, 17: 7, 1989.

Other methods useful for detecting variations (e.g., substitutions) include, but are not limited to: (1) mutation-specific nucleotide incorporation assays, such as single base extension assays (see, e.g., Chen et al. (2000) Genome Res. 10: 549-557); (2) mutation-specific primer extension assays (see, e.g., Ye et al. (2001) Hum. Mut. 17: 305-316); (3) 5 ′nuclease assay (see, e.g., De La Vega et al. (2002) BioTechniques 32: S48-S54 (which describes the TaqMan® assay); (4) assays using molecular pathway labels (see, e.g., Tyagi et al. (1998) Nature Biotech. 16: 49-53); (5) oligonucleotide ligation assays (see, e.g., Grossman et al. (1994) Nuc. Acids Res. 22: 4527-4534) and (6) allele-specific PCR;

Variations can also be detected by mismatch detection methods. Mismatches are hybridized nucleic acid duplexes that are not 100% complementary. Lack of total complementarity can be attributed to deletions, insertions, inversions, or substitutions. One example of a mismatch detection method is, for example, a mismatch recovery detection (MRD) assay described in Faham et al., Proc. Natl. Acad. Sci. USA 102: 14717-14722 (2005). Another example of a mismatched cutting technique is the RNase protection method described in detail in Myers et al., Science 230: 1242, 1985. For example, the methods used to detect variation may include the use of labeled riboprobes that are complementary to human wild type target nucleic acids. Riboprobes and target nucleic acids derived from tissue samples are annealed (hybridized) together and subsequently digested with the enzyme RNase A, which can detect some mismatches in the duplex RNA structure. If a mismatch is detected by RNase A, it is cleaved at the site of the mismatch. Thus, when annealed RNA preparations are separated on an electrophoretic gel matrix, if mismatches are detected and cleaved by RNase A, smaller RNA products will be observed than mRNA or full length duplex RNA for DNA and riboprobes. Riboprobes need not be the full length of the target nucleic acid, but can be part of the target nucleic acid, as long as it includes a position suspected of having a mutation.

In a similar manner, DNA probes can be used to detect mismatches, for example, via enzymatic or chemical cleavage. For example, Cotton et al., Proc. Natl. Acad. Sci. USA, 85: 4397, 1988. Alternatively, discrepancies can be detected by the transition of the electrophoretic mobility of the mismatched duplex to the matched duplex. See, e.g., Cariello, Human Genetics, 42: 726, 1988. With either riboprobes or DNA probes, target nucleic acids suspected of containing mutations can be amplified prior to hybridization. In particular, if the change is a severe rearrangement such as deletion and insertion, changes in the target nucleic acid can also be detected using Southern hybridization.

Restriction fragment length polymorphism (RFLP) probes to target nucleic acids or surrounding marker genes can be used to detect variations, for example insertions or deletions. Insertions and deletions can also be detected by cloning, sequencing and amplification of target nucleic acids. Single stranded polymorphism (SSCP) assays can also be used to detect base altering variants of the allele. SSCP can be modified for the detection of ErbB2 somatic mutations. SSCP identifies base differences due to alterations in electrophoretic shifting of single stranded PCR products. Single-stranded PCR products can be produced by heating or otherwise denaturing the double-stranded PCR product. Single-stranded nucleic acids may refold or form secondary structures that are partially dependent on the base sequence. Different electrophoretic mobility of single-stranded amplification products is related to base-sequence differences at SNP positions. Denaturation gradient gel electrophoresis (DGGE) differentiates SNP alleles based on different sequence-dependent stability and melting characteristics inherent to polymorphic DNA and corresponding differences in electrophoretic migration patterns in denaturing gradient gels.

Somatic mutations or modifications can also be detected using microarrays. Microarrays are typically a multiplex technique using a series of thousands of nucleic acid probes arranged to hybridize under high-stringency conditions, e.g., with a cDNA or cRNA sample. Probe-target hybridization is typically detected and quantified by detection of fluorophore-, silver-, or chemiluminescent-labeled targets to determine the relative abundance of nucleic acid sequences at the target. In a typical microarray, the probe is attached to a hard surface by covalent bonds to the chemical matrix (via epoxy-silane, amino-silane, lysine, polyacrylamide or the like). Hard surfaces are, for example, glass, silicon chips, or microscopic beads.

Another method for the detection of somatic mutations is based on mass spectrometry. Mass spectrometry uses the unique mass of each of the four nucleotides of DNA. Potential mutation-containing ErbB2 nucleic acids can be clearly analyzed by mass spectrometry by measuring the difference in mass of nucleic acids with somatic mutations. MALDI-TOF (matrix assisted laser desorption ionization-timeout) mass spectrometry techniques are useful for extremely accurate determination of molecular weight, such as nucleic acids containing somatic mutations. Numerous approaches to nucleic acid analysis have been developed based on mass spectrometry. Exemplary mass spectrometry-based methods also include primer extension assays, which can be used in combination with other approaches, such as traditional gel-based formats and microarrays.

Sequence-specific ribozymes (U.S. Pat. No. 5,498,531) can also be used to detect somatic mutations based on the development or loss of ribozyme cleavage sites. Perfectly matched sequences can be distinguished from mismatched sequences by nuclease cleavage digestion assays or differences in melting temperatures. If a mutation affects a restriction enzyme cleavage site, the mutation can be identified by a change in the restriction enzyme digestion pattern and a corresponding change in nucleic acid fragment length determined by gel electrophoresis.

In certain embodiments of the present disclosure, protein-based detection techniques are used to detect variant proteins encoded by genes with genetic variations as disclosed herein. Determination of the presence of variant forms of proteins can be performed by any suitable technique known in the art, for example electrophoresis (e.g., denatured or non-modified polyacrylamide gel electrophoresis, two-dimensional gel electrophoresis, capillary electrophoresis). Electrophoresis, and isoelectronic focusing, chromatography (e.g., sizing chromatography, high performance liquid chromatography (HPLC), and cation exchange HPLC), mass spectroscopy (e.g., MALDI-TOF mass spectroscopy, electrospray), ionization (ESI) mass spectroscopy, and tandem mass spectroscopy). See, e.g., Ahrer and Jungabauer (2006) J. Chromatog. B. Analyt. Technol. Biomed. Life Sci. 841: 110-122. A suitable technique can be selected based in part on the nature of the variation detected. For example, variations in which substituted amino acids result in amino acid substitutions with charges different from the original amino acids can be detected by isoelectric point electrophoresis. Isoelectric electrophoresis of a polypeptide through a gel with a pH gradient at high voltage separates the protein by its isoelectric point (pi). pH gradient gels can be compared to co-operated gels containing wild type protein. In instances where the mutation results in the generation of new proteolytic cleavage sites or the abolition of existing ones, the samples can be peptide mapped using proteolytic digestion followed by appropriate electrophoresis, chromatography, or mass spectrometry techniques. The presence of the variation can also be detected using protein sequencing techniques such as Edman degradation or certain forms of mass spectroscopy.

Methods known in the art using a combination of these techniques can also be used. For example, in HPLC-microscopy tandem mass spectrometry techniques, proteolytic digestion is performed on proteins and the resulting peptide mixtures are separated by reverse phase chromatography separation. Tandem mass spectrometry is then performed and the data collected therefrom are analyzed. In another example, unmodified gel electrophoresis is combined with MALDI mass spectroscopy

In certain embodiments, a protein can be isolated from a sample using reagents such as antibodies or peptides that specifically bind to the protein, and then further analyzed to present the genetic variation using any of the techniques disclosed above.

Alternatively, the presence of the variant protein in the sample may be directed to an antibody specific for a protein having a genetic variation, i.e., an antibody that specifically binds to a protein having a mutation but does not bind to a protein having no mutation. It can be detected by an immunoaffinity assay. Such antibodies can be produced by any suitable technique known in the art. Antibodies can be used to immunoprecipitate a particular protein from a solution sample or to immunoblot a protein separated by, for example, a polyacrylamide gel. Immunocytochemical methods can also be used to detect specific protein variants in tissues or cells. For example, immunoenzymatic assays (IEMA), including enzyme-linked immunosorbent assays (ELISA), radioimmunoassay (RIA), immunoradiometric (IRMA) and sandwich assays using monoclonal or polyclonal antibodies.

B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day).

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering antibodies and/or antibody-drug conjugate of the present invention are well known in the art and may be selected by those of ordinary skill in the art. In one embodiment, tucatinib, or salt or solvate thereof, administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib, or salt or solvate thereof, is intravenous injection or infusion. In some embodiments, the route of administration of tucatinib, or salt or solvate thereof, is intravenous infusion. In some embodiments, the route of administration of tucatinib, or salt or solvate thereof, is intravenous injection or infusion. In some embodiments, the tucatinib, or salt or solvate thereof, is intravenous infusion. In some embodiments, the route of administration of tucatinib, or salt or solvate thereof, is oral.

In one embodiment of the methods or uses or product for uses provided herein, tucatinib, or salt or solvate thereof, is administered to the subject daily, twice daily, three times daily or four times daily. In some embodiments, tucatinib, or salt or solvate thereof, is administered to the subject every other day, once about every week or once about every three weeks. In some embodiments, tucatinib, or salt or solvate thereof, is administered to the subject once per day. In some embodiments, tucatinib, or salt or solvate thereof, is administered to the subject twice per day. In some embodiments, tucatinib, or salt or solvate thereof, is administered to the subject at a dose of about 300 mg twice per day. In some embodiments, tucatinib, or salt or solvate thereof, is administered to the subject at a dose of 300 mg twice per day. In some embodiments, tucatinib, or salt or solvate thereof, is administered to the subject at a dose of about 600 mg once per day. In some embodiments, tucatinib, or salt or solvate thereof, is administered to the subject at a dose of 600 mg once per day. In some embodiments, tucatinib, or salt or solvate thereof, is administered to the subject twice per day on each day of a 21-day treatment cycle. In some embodiments, the tucatinib, or salt or solvate thereof, is administered to the subject orally.

C. Anti-HER2 Antibody Dose and Administration

In some embodiments, a dose of the anti-HER2 antibody is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of the anti-HER2 antibody is between about 4 mg and 10 mg per kg of the subject's body weight. In some embodiments, a dose of the anti-HER2 antibody is between 4 mg and 10 mg per kg of the subject's body weight. In some embodiments, a dose of the anti-HER2 antibody is about 6 mg per kg of the subject's body weight. In some embodiments, a dose of the anti-HER2 antibody is about 8 mg per kg of the subject's body weight. In some embodiments, a dose of the anti-HER2 antibody is about 8 mg per kg of the subject's body weight for the first dose of the anti-HER2 antibody administered to the subject followed by subsequent doses of about 6 mg per kg of the subject's body weight. In some embodiments, a dose of the anti-HER2 antibody is 6 mg per kg of the subject's body weight. In some embodiments, a dose of the anti-HER2 antibody is 8 mg per kg of the subject's body weight. In some embodiments, a dose of the anti-HER2 antibody is 8 mg per kg of the subject's body weight for the first dose of the anti-HER2 antibody administered to the subject followed by subsequent doses of 6 mg per kg of the subject's body weight. In other embodiments, a dose of the anti-HER2 antibody is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of the anti-HER2 antibody is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of the anti-HER2 antibody is about 6 mg per kg of the subject's body weight. In other instances, a dose of the anti-HER2 antibody is about 8 mg per kg of the subject's body weight. In some other instances, a dose of the anti-HER2 antibody is about 20 mg per kg of the subject's body weight. In some embodiments, a dose of the anti-HER2 antibody comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the anti-HER2 antibody. In other embodiments, a dose of the anti-HER2 antibody comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the anti-HER2 antibody. In particular embodiments, a dose of the anti-HER2 antibody comprises between about 100 mg and 400 mg (e.g., about 100, 125, 150, 175, 200, 225, 250, 275, 300, 325, 350, 375, or 400 mg) of the anti-HER2 antibody. In some embodiments, a dose of the anti-HER2 antibody is between about 400 mg and 800 mg. In some embodiments, a dose of the anti-HER2 antibody is between 400 mg and 800 mg. In some embodiments, a dose of the anti-HER2 antibody is about 600 mg. In some embodiments, a dose of the anti-HER2 antibody is 600 mg. As a non-limiting example, when using a dose of 6 mg/kg, a dose for a 50 kg subject will be about 300 mg. As another non-limiting example, when using a dose of 8 mg/kg, a dose for a 50 kg subject will be about 400 mg. In some embodiments, a dose of the anti-HER2 antibody comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the anti-HER2 antibody. In some embodiments, a dose of the anti-HER2 antibody contains a therapeutically effective amount of the anti-HER2 antibody. In other embodiments, a dose of the anti-HER2 antibody contains less than a therapeutically effective amount of the anti-HER2 antibody (e.g., when multiple doses are given in order to achieve the desired clinical or therapeutic effect). In some embodiments, the anti-HER2 antibody is administered to the subject once about every 1 to 4 weeks. In certain embodiments, an anti-HER2 antibody is administered once about every 1 week, once about every 2 weeks, once about every 3 weeks or once about every 4 weeks. In one embodiment, an anti-HER2 antibody is administered once about every 3 weeks. In some embodiments, the anti-HER2 antibody is administered to the subject once every 1 to 4 weeks. In certain embodiments, an anti-HER2 antibody is administered once every 1 week, once about every 2 weeks, once about every 3 weeks or once about every 4 weeks. In one embodiment, an anti-HER2 antibody is administered once every 3 weeks. In some embodiments, the anti-HER2 antibody is administered to the subject subcutaneously. In some embodiments, the anti-HER2 antibody is administered to the subject intraperitoneally. In some embodiments, the anti-HER2 antibody is administered to the subject intravenously. In some embodiments, the at least one anti-HER2 antibody is one anti-HER2 antibody. In some embodiments, the at least one anti-HER2 antibody is a combination of two anti-HER2 antibodies. In some embodiments, the at least one anti-HER2 antibody is a combination of three anti-HER2 antibodies. In some embodiments, the at least one anti-HER2 antibody is a combination of four anti-HER2 antibodies. In some embodiments, the anti-HER2 antibody is selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab. In some embodiments, the anti-HER2 antibody is pertuzumab. In some embodiments, the anti-HER2 antibody is administered at a dose of about 600 mg once about every 3 weeks and the anti-HER2 antibody is administered subcutaneously. In some embodiments, the anti-HER2 antibody is administered at a dose of 600 mg once every 3 weeks and the anti-HER2 antibody is administered subcutaneously. In some embodiments, the anti-HER2 antibody is trastuzumab and is administered at a dose of about 600 mg once about every 3 weeks and the trastuzumab is administered subcutaneously. In some embodiments, the anti-HER2 antibody is trastuzumab and is administered at a dose of 600 mg once every 3 weeks and the trastuzumab is administered subcutaneously. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21-day treatment cycle and is administered to the subject once per treatment cycle. In some embodiments, the anti-HER2 antibody is trastuzumab and is administered to the subject on day one of a 21-day treatment cycle and is administered to the subject once per treatment cycle.

In some embodiments, the at least one anti-HER2 antibody comprises a first anti-HER2 antibody and a second anti-HER2 antibody. In some embodiments, a dose of the first anti-HER2 antibody is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of the first anti-HER2 antibody is between about 4 mg and 10 mg per kg of the subject's body weight. In some embodiments, a dose of the first anti-HER2 antibody is between 4 mg and 10 mg per kg of the subject's body weight. In some embodiments, a dose of the first anti-HER2 antibody is about 6 mg per kg of the subject's body weight. In some embodiments, a dose of the first anti-HER2 antibody is about 8 mg per kg of the subject's body weight. In some embodiments, a dose of the first anti-HER2 antibody is about 8 mg per kg of the subject's body weight for the first dose of the first anti-HER2 antibody administered to the subject followed by subsequent doses of about 6 mg per kg of the subject's body weight. In some embodiments, a dose of the first anti-HER2 antibody is 6 mg per kg of the subject's body weight. In some embodiments, a dose of the first anti-HER2 antibody is 8 mg per kg of the subject's body weight. In some embodiments, a dose of the first anti-HER2 antibody is 8 mg per kg of the subject's body weight for the first dose of the first anti-HER2 antibody administered to the subject followed by subsequent doses of 6 mg per kg of the subject's body weight. In other embodiments, a dose of the first anti-HER2 antibody is between about 10 mg and 100 mg per kg of the subject's body weight (e.g., about 10, 11, 12, 13, 14,15,16,17,18, 19,20,21,22,23,24,25,26,27,28,29,30,31,32,33,34,35,36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of the first anti-HER2 antibody is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of the first anti-HER2 antibody is about 6 mg per kg of the subject's body weight. In other instances, a dose of the first anti-HER2 antibody is about 8 mg per kg of the subject's body weight. In some other instances, a dose of the first anti-HER2 antibody is about 20 mg per kg of the subject's body weight. In some embodiments, a dose of the first anti-HER2 antibody comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the first anti-HER2 antibody. In other embodiments, a dose of the first anti-HER2 antibody comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the first anti-HER2 antibody. In particular embodiments, a dose of the first anti-HER2 antibody comprises between about 100 mg and 400 mg (e.g., about 100, 125, 150, 175, 200, 225, 250, 275, 300, 325, 350, 375, or 400 mg) of the first anti-HER2 antibody. In some embodiments, a dose of the first anti-HER2 antibody is between about 400 mg and 800 mg. In some embodiments, a dose of the first anti-HER2 antibody is between 400 mg and 800 mg. In some embodiments, a dose of the first anti-HER2 antibody is about 600 mg. In some embodiments, a dose of the first anti-HER2 antibody is 600 mg. In some embodiments, a dose of the first anti-HER2 antibody comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the first anti-HER2 antibody. In some embodiments, a dose of the first anti-HER2 antibody contains a therapeutically effective amount of the first anti-HER2 antibody. In other embodiments, a dose of the first anti-HER2 antibody contains less than a therapeutically effective amount of the first anti-HER2 antibody (e.g., when multiple doses are given in order to achieve the desired clinical or therapeutic effect). In some embodiments, the first anti-HER2 antibody is administered to the subject once about every 1 to 4 weeks. In certain embodiments, the first anti-HER2 antibody is administered once about every 1 week, once about every 2 weeks, once about every 3 weeks or once about every 4 weeks. In one embodiment, the first anti-HER2 antibody is administered once about every 3 weeks. In some embodiments, the first anti-HER2 antibody is administered to the subject once every 1 to 4 weeks. In certain embodiments, the first anti-HER2 antibody is administered once every 1 week, once about every 2 weeks, once about every 3 weeks or once about every 4 weeks. In one embodiment, the first anti-HER2 antibody is administered once every 3 weeks. In some embodiments, the first anti-HER2 antibody is administered to the subject subcutaneously. In some embodiments, the first anti-HER2 antibody is administered to the subject intraperitoneally. In some embodiments, the first anti-HER2 antibody is administered to the subject intravenously. In some embodiments, the first anti-HER2 antibody is selected from the group consisting of trastuzumab, pertuzumab, ado-trastuzumab emtansine, and margetuximab. In some embodiments, the first anti-HER2 antibody is trastuzumab. In some embodiments, the first anti-HER2 antibody is administered at a dose of about 600 mg once about every 3 weeks and the first anti-HER2 antibody is administered subcutaneously. In some embodiments, the first anti-HER2 antibody is administered at a dose of 600 mg once every 3 weeks and the first anti-HER2 antibody is administered subcutaneously. In some embodiments, the first anti-HER2 antibody is trastuzumab and is administered at a dose of about 600 mg once about every 3 weeks and the trastuzumab is administered subcutaneously. In some embodiments, the first anti-HER2 antibody is trastuzumab and is administered at a dose of 600 mg once every 3 weeks and the trastuzumab is administered subcutaneously. In some embodiments, the first anti-HER2 antibody is administered at a dose of about 6 mg/kg once about every 3 weeks and the first anti-HER2 antibody is administered intravenously. In some embodiments, the first anti-HER2 antibody is administered at a dose of about 8 mg/kg once about every 3 weeks and the first anti-HER2 antibody is administered intravenously. In some embodiments, the first anti-HER2 antibody is administered once about every 3 weeks at a dose of about 8 mg/kg for the first dose of the first anti-HER2 antibody administered to the subject followed by subsequent doses of about 6 mg/kg, wherein first anti-HER2 antibody is administered intravenously. In some embodiments, the first anti-HER2 antibody is administered at a dose of 6 mg/kg once every 3 weeks and the first anti-HER2 antibody is administered intravenously. In some embodiments, the first anti-HER2 antibody is administered at a dose of 8 mg/kg once every 3 weeks and the first anti-HER2 antibody is administered intravenously. In some embodiments, the first anti-HER2 antibody is administered once every 3 weeks at a dose of 8 mg/kg for the first dose of the first anti-HER2 antibody administered to the subject followed by subsequent doses of 6 mg/kg, wherein first anti-HER2 antibody is administered intravenously. In some embodiments, the first anti-HER2 antibody is trastuzumab and is administered at a dose of about 6 mg/kg once about every 3 weeks and the trastuzumab is administered intravenously. In some embodiments, the first anti-HER2 antibody is trastuzumab and is administered at a dose of about 8 mg/kg once about every 3 weeks and the trastuzumab is administered intravenously. In some embodiments, the first anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the first anti-HER2 antibody is trastuzumab and is administered at a dose of 6 mg/kg once every 3 weeks and the trastuzumab is administered intravenously. In some embodiments, the first anti-HER2 antibody is trastuzumab and is administered at a dose of 8 mg/kg once every 3 weeks and the trastuzumab is administered intravenously. In some embodiments, the first anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the first anti-HER2 antibody is trastuzumab and is administered to the subject on a 21-day treatment cycle and is administered to the subject once per treatment cycle. In some embodiments, the first anti-HER2 antibody is trastuzumab and is administered to the subject on day one of a 21-day treatment cycle and is administered to the subject once per treatment cycle.

In some embodiments, the at least one anti-HER2 antibody comprises a first anti-HER2 antibody and a second anti-HER2 antibody. In some embodiments, a dose of the second anti-HER2 antibody is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of the second anti-HER2 antibody is between about 4 mg and 10 mg per kg of the subject's body weight. In some embodiments, a dose of the second anti-HER2 antibody is between 4 mg and 10 mg per kg of the subject's body weight. In some embodiments, a dose of the second anti-HER2 antibody is about 6 mg per kg of the subject's body weight. In some embodiments, a dose of the second anti-HER2 antibody is about 8 mg per kg of the subject's body weight. In some embodiments, a dose of the second anti-HER2 antibody is about 8 mg per kg of the subject's body weight for the first dose of the second anti-HER2 antibody administered to the subject followed by subsequent doses of about 6 mg per kg of the subject's body weight. In some embodiments, a dose of the second anti-HER2 antibody is 6 mg per kg of the subject's body weight. In some embodiments, a dose of the second anti-HER2 antibody is 8 mg per kg of the subject's body weight. In some embodiments, a dose of the second anti-HER2 antibody is 8 mg per kg of the subject's body weight for the first dose of the second anti-HER2 antibody administered to the subject followed by subsequent doses of 6 mg per kg of the subject's body weight. In other embodiments, a dose of the second anti-HER2 antibody is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of the second anti-HER2 antibody is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of the second anti-HER2 antibody is about 6 mg per kg of the subject's body weight. In other instances, a dose of the second anti-HER2 antibody is about 8 mg per kg of the subject's body weight. In some other instances, a dose of the second anti-HER2 antibody is about 20 mg per kg of the subject's body weight. In some embodiments, a dose of the second anti-HER2 antibody comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the second anti-HER2 antibody. In other embodiments, a dose of the second anti-HER2 antibody comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the second anti-HER2 antibody. In particular embodiments, a dose of the second anti-HER2 antibody comprises between about 100 mg and 400 mg (e.g., about 100, 125, 150, 175, 200, 225, 250, 275, 300, 325, 350, 375, or 400 mg) of the second anti-HER2 antibody. In some embodiments, a dose of the second anti-HER2 antibody is between about 400 mg and 800 mg. In some embodiments, a dose of the second anti-HER2 antibody is between 400 mg and 800 mg. In some embodiments, a dose of the second anti-HER2 antibody is about 600 mg. In some embodiments, a dose of the second anti-HER2 antibody is 600 mg. In some embodiments, a dose of the second anti-HER2 antibody comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the second anti-HER2 antibody. In some embodiments, a dose of the second anti-HER2 antibody contains a therapeutically effective amount of the second anti-HER2 antibody. In other embodiments, a dose of the second anti-HER2 antibody contains less than a therapeutically effective amount of the second anti-HER2 antibody (e.g., when multiple doses are given in order to achieve the desired clinical or therapeutic effect). In some embodiments, the second anti-HER2 antibody is administered to the subject once about every 1 to 4 weeks. In certain embodiments, the second anti-HER2 antibody is administered once about every 1 week, once about every 2 weeks, once about every 3 weeks or once about every 4 weeks. In one embodiment, the second anti-HER2 antibody is administered once about every 3 weeks. In some embodiments, the second anti-HER2 antibody is administered to the subject once every 1 to 4 weeks. In certain embodiments, the second anti-HER2 antibody is administered once every 1 week, once about every 2 weeks, once about every 3 weeks or once about every 4 weeks. In one embodiment, the second anti-HER2 antibody is administered once every 3 weeks. In some embodiments, the second anti-HER2 antibody is administered to the subject subcutaneously. In some embodiments, the second anti-HER2 antibody is administered to the subject intraperitoneally. In some embodiments, the second anti-HER2 antibody is administered to the subject intravenously. In some embodiments, the second anti-HER2 antibody is selected from the group consisting of trastuzumab, pertuzumab, ado-trastuzumab emtansine, and margetuximab. In some embodiments, the second anti-HER2 antibody is pertuzumab. In some embodiments, the second anti-HER2 antibody is administered at a dose of about 600 mg once about every 3 weeks and the second anti-HER2 antibody is administered subcutaneously. In some embodiments, the second anti-HER2 antibody is administered at a dose of 600 mg once every 3 weeks and the second anti-HER2 antibody is administered subcutaneously. In some embodiments, the second anti-HER2 antibody is pertuzumab and is administered at a dose of about 600 mg once about every 3 weeks and the pertuzumab is administered subcutaneously. In some embodiments, the second anti-HER2 antibody is pertuzumab and is administered at a dose of 600 mg once every 3 weeks and the pertuzumab is administered subcutaneously. In some embodiments, the second anti-HER2 antibody is administered at a dose of about 6 mg/kg once about every 3 weeks and the second anti-HER2 antibody is administered intravenously. In some embodiments, the second anti-HER2 antibody is administered at a dose of about 8 mg/kg once about every 3 weeks and the second anti-HER2 antibody is administered intravenously. In some embodiments, the second anti-HER2 antibody is administered once about every 3 weeks at a dose of about 8 mg/kg for the first dose of the second anti-HER2 antibody administered to the subject followed by subsequent doses of about 6 mg/kg, wherein second anti-HER2 antibody is administered intravenously. In some embodiments, the second anti-HER2 antibody is administered at a dose of 6 mg/kg once every 3 weeks and the second anti-HER2 antibody is administered intravenously. In some embodiments, the second anti-HER2 antibody is administered at a dose of 8 mg/kg once every 3 weeks and the second anti-HER2 antibody is administered intravenously. In some embodiments, the second anti-HER2 antibody is administered once every 3 weeks at a dose of 8 mg/kg for the first dose of the second anti-HER2 antibody administered to the subject followed by subsequent doses of 6 mg/kg, wherein second anti-HER2 antibody is administered intravenously. In some embodiments, the second anti-HER2 antibody is pertuzumab and is administered at a dose of about 6 mg/kg once about every 3 weeks and the pertuzumab is administered intravenously. In some embodiments, the second anti-HER2 antibody is pertuzumab and is administered at a dose of about 8 mg/kg once about every 3 weeks and the pertuzumab is administered intravenously. In some embodiments, the second anti-HER2 antibody is pertuzumab and is administered once about every 3 weeks at a dose of about 8 mg/kg for the first dose of the pertuzumab administered to the subject followed by subsequent doses of about 6 mg/kg, wherein the pertuzumab is administered intravenously. In some embodiments, the second anti-HER2 antibody is pertuzumab and is administered at a dose of 6 mg/kg once every 3 weeks and the pertuzumab is administered intravenously. In some embodiments, the second anti-HER2 antibody is pertuzumab and is administered at a dose of 8 mg/kg once every 3 weeks and the pertuzumab is administered intravenously. In some embodiments, the second anti-HER2 antibody is pertuzumab and is administered once every 3 weeks at a dose of 8 mg/kg for the first dose of pertuzumab administered to the subject followed by subsequent doses of 6 mg/kg, wherein the pertuzumab is administered intravenously. In some embodiments, the second anti-HER2 antibody is pertuzumab and is administered to the subject on a 21-day treatment cycle and is administered to the subject once per treatment cycle. In some embodiments, the second anti-HER2 antibody is pertuzumab and is administered to the subject on day one of a 21-day treatment cycle and is administered to the subject once per treatment cycle.

In some embodiments, a method of treatment described herein comprises administering to the subject tucatinib, or salt or solvate thereof, and trastuzumab. In some embodiments, the tucatinib, or salt or solvate thereof, and trastuzumab are administered to the subject on a 21-day treatment cycle. In some embodiments, tucatinib, or salt or solvate thereof, is administered to the subject at a dose of about 300 mg twice per day. In some embodiments, tucatinib, or salt or solvate thereof, is administered to the subject at a dose of 300 mg twice per day. In some embodiments, tucatinib, or salt or solvate thereof, is administered to the subject at a dose of about 600 mg once per day. In some embodiments, tucatinib, or salt or solvate thereof, is administered to the subject at a dose of 600 mg once per day. In some embodiments, tucatinib, or salt or solvate thereof, is administered to the subject twice per day on each day of a 21-day treatment cycle. In some embodiments, the tucatinib, or salt or solvate thereof, is administered to the subject orally. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21-day treatment cycle and is administered to the subject once per treatment cycle. In some embodiments, the anti-HER2 antibody is trastuzumab and is administered to the subject on day one of a 21-day treatment cycle and is administered to the subject once per treatment cycle.

In some embodiments, a method of treatment described herein comprises administering to the subject tucatinib, or salt or solvate thereof, and at least one anti-HER2 antibody. In some embodiments, a method of treatment described herein comprises administering to the subject tucatinib, or salt or solvate thereof, and at least one anti-HER2 antibody, wherein the at least one anti-HER2 antibody comprises a first anti-HER2 antibody and a second anti-HER2 antibody, wherein the first anti-HER2 antibody is trastuzumab and the second anti-HER2 antibody is pertuzumab. In some embodiments, the tucatinib, or salt or solvate thereof, the trastuzumab, and the pertuzumab are administered to the subject on a 21-day treatment cycle. In some embodiments, tucatinib, or salt or solvate thereof, is administered to the subject at a dose of about 300 mg twice per day. In some embodiments, tucatinib, or salt or solvate thereof, is administered to the subject at a dose of 300 mg twice per day. In some embodiments, tucatinib, or salt or solvate thereof, is administered to the subject at a dose of about 600 mg once per day. In some embodiments, tucatinib, or salt or solvate thereof, is administered to the subject at a dose of 600 mg once per day. In some embodiments, tucatinib, or salt or solvate thereof, is administered to the subject twice per day on each day of a 21-day treatment cycle. In some embodiments, the tucatinib, or salt or solvate thereof, is administered to the subject orally. In some embodiments, the first anti-HER2 antibody is administered at a dose of about 6 mg/kg once about every 3 weeks and the first anti-HER2 antibody is administered intravenously. In some embodiments, the first anti-HER2 antibody is administered at a dose of about 600 mg once about every 3 weeks and the first anti-HER2 antibody is administered subcutaneously. In some embodiments, the second anti-HER2 antibody is administered at a dose of about 420 mg once about every 3 weeks and the second anti-HER2 antibody is administered intravenously. In some embodiments, the second anti-HER2 antibody is administered at a dose of about 600 mg once about every 3 weeks and the second anti-HER2 antibody is administered subcutaneously. In some embodiments, the first anti-HER2 antibody and the second anti-HER2 antibody are administered together as a pharmaceutical composition. In some embodiments, the first anti-HER2 antibody and the second anti-HER2 antibody are administered together as a pharmaceutical composition comprising about 600 mg of the first anti-HER2 antibody and about 600 mg of the second anti-HER2 antibody. In some embodiments, the pharmaceutical composition further comprises hyaluronidase. In some embodiments, the pharmaceutical composition comprises about 20,000 units hyaluronidase. In some embodiments, the pharmaceutical composition is administered subcutaneously. In some embodiments, the pharmaceutical composition is administered subcutaneously about once every 3 weeks.

In an exemplary embodiment, a method of treatment described herein comprises administering trastuzumab at a dose of about 6 mg/kg intravenously about once every 3 weeks and administering pertuzumab at a dose of about 420 mg intravenously about once every 3 weeks.

In an exemplary embodiment, a method of treatment described herein comprises administering trastuzumab at a dose of about 600 mg subcutaneously about once every 3 weeks and administering pertuzumab at a dose of about 420 mg intravenously about once every 3 weeks.

In an exemplary embodiment, a method of treatment described herein comprises administering a pharmaceutical composition subcutaneously about once every 3 weeks, wherein the pharmaceutical composition comprises about 600 mg trastuzumab and about 600 mg pertuzumab.

In an exemplary embodiment, a method of treatment described herein comprises administering a pharmaceutical composition subcutaneously about once every 3 weeks, wherein the pharmaceutical composition comprises about 600 mg trastuzumab, about 600 mg pertuzumab, and about 20,000 units hyaluronidase.

D. Fulvestrant Dose and Administration

In some embodiments, the methods of treatment described herein are methods of treating breast cancer in a subject. In some embodiments, the breast cancer is hormone receptor (HR) positive (HR+) breast cancer. In some embodiments, the HR+ breast cancer is HER-2 mutated breast cancer. In some embodiments, the subject is administered fulvestrant in combination with tucatinib, or salt or solvate thereof, as described herein and at least one anti-HER2 antibody as described herein. Fulvestrant (Faslodex®) is an estrogen receptor (ER) antagonist approved for use in treatment of hormone receptor (HR) positive (HR+) metastatic breast cancer (mBC) in postmenopausal women with disease progression following antiestrogen therapy. In some embodiments, the subject is administered fulvestrant in combination with tucatinib, or salt or solvate thereof, and trastuzumab. In some embodiments, the fulvestrant is administered at a dose between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1.000 mg) of fulvestrant. In one embodiment, fulvestrant is administered at a dose of about 500 mg. In one embodiment, fulvestrant is administered at a dose of 500 mg. In some embodiments, fulvestrant is administered once about every 4 weeks. In some embodiments, fulvestrant is administered once every 4 weeks. In some embodiments, fulvestrant is administered once every 4 weeks stating on Day 1 of the first 21-day treatment cycle. In some embodiments, Day 1 of the first 21-day treatment cycle is the day of the first administration of trastuzumab. In some embodiments, fulvestrant is administered once every 4 weeks stating on Day 1 of the first 21-day treatment cycle, as well as on Day 15 of the first 21-day treatment cycle. In some embodiments, fulvestrant is administered at a dose of 500 mg once every 4 weeks stating on Day 1 of the first 21-day treatment cycle, as well as on day 15 of the first 21-day treatment cycle. In some embodiments, fulvestrant is administered intramuscularly. In some embodiments, fulvestrant is administered intramuscularly at a dose of 500 mg once every 4 weeks stating on Day 1 of the first 21-day treatment cycle, as well as on day 15 of the first 21-day treatment cycle.

E. Treatment Outcome

In some embodiments, treating the subject comprises inhibiting cancer cell growth, inhibiting cancer cell proliferation, inhibiting cancer cell migration, inhibiting cancer cell invasion, decreasing or eliminating one or more signs or symptoms of cancer, reducing the size (e.g., volume) of a cancer tumor, reducing the number of cancer tumors, reducing the number of cancer cells, inducing cancer cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%, 104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib and trastuzumab results in a TGI index that is greater than the TGI index that is observed when tucatinib or trastuzumab is used alone. In some instances, treating the subject results in a TGI index that is greater than the TGI index that is observed when tucatinib is used alone. In other instances, treating the subject results in a TGI index that is greater than the TGI index that is observed when trastuzumab is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib or trastuzumab is used alone.

In some embodiments, the combination of the tucatinib and trastuzumab is synergistic. In particular embodiments, with respect to the synergistic combination, treating the subject results in a TGI index that is greater than the TGI index that would be expected if the combination of tucatinib and trastuzumab produced an additive effect. In some instances, the TGI index observed when a combination of tucatinib and trastuzumab is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib and trastuzumab produced an additive effect.

In some embodiments, treating the subject as described herein results in an increase in the overall amount of HER2 in the solid tumor. In some embodiments, the amount of HER2 in the solid tumor is determined by western blot analysis. In some embodiments, the amount of HER2 in the solid tumor is determined by immunohistochemistry. In some embodiments, the amount of HER2 in the solid tumor is determined by mass spectrometry. In some embodiments, the amount of HER2 in the solid tumor is determined by ELISA. In some embodiments, the amount of HER2 in the solid tumor is determined by real-time quantitative PCR (qRT-PCR). In some embodiments, the amount of HER2 in the solid tumor is determined by microarray analysis. In some embodiments, treating the subject as described herein results in an increase in the amount of plasma membrane-bound HER2 in the solid tumor. In some embodiments, the amount of plasma membrane-bound HER2 in the solid tumor is determined by quantitative fluorescence activated cell sorting (qFACS). In some embodiments, treating the subject as described herein results in an increase in the dwell time of HER2 at the cell surface. In some embodiments, treating the subject as described herein results in an increase in the internalization of plasma membrane-bound HER2. In some embodiments, treating the subject as described herein results in an increase in the lysosomal degradation of HER2.

In one aspect, a method of treating cancer with tucatinib as described herein and at least one anti-HER2 antibody as described herein results in an improvement in one or more therapeutic effects in the subject after administration of tucatinib as described herein and the at least one anti-HER2 antibody as described herein relative to a baseline. In some embodiments, the one or more therapeutic effects is the size of the tumor derived from the solid tumor, the objective response rate, the duration of response, the time to response, progression free survival, overall survival, or any combination thereof. In one embodiment, the one or more therapeutic effects is the size of the tumor derived from the solid tumor. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with tucatinib as described herein and at least one anti-HER2 antibody as described herein may include the following criteria (RECIST Criteria 1.1):

| | Category | Criteria |
|---|---|---|
| Based | Complete | Disappearance of all target lesions. Any |
| on | Response | pathological lymph nodes must have reduction |
| target | (CR) | in short axis to <10 mm. |
| lesions | Partial | ≥30% decrease in the sum of the longest |
| | Response | diameter (LD) of target lesions, taking as |
| | (PR) | reference the baseline sum of LDs. |
| | Stable | Neither sufficient shrinkage to qualify for PR nor |
| | Disease | sufficient increase to qualify for PD, taking as |
| | (SD) | reference the smallest sum of LDs while in trial. |
| | Progressive | ≥20% (and ≥5 mm) increase in the sum of the |
| | Disease (PD) | LDs of target lesions, taking as reference the |
| | | smallest sum of the target LDs recorded while in |
| | | trial or the appearance of one or more new |
| | | lesions. |
| Based | CR | Disappearance of all non-target lesions and |
| on non- | | normalization of tumor marker level. All lymph |
| target | | nodes must be non-pathological in size (<10 mm |
| lesions | | short axis). |
| | SD | Persistence of one or more non-target lesion(s) |
| | | or/and maintenance of tumor marker level above |
| | | the normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with tucatinib described herein and at least one anti-HER2 antibody described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with tucatinib described herein and at least one anti-HER2 antibody described herein is assessed by measuring the size of a tumor derived from the cancer described herein (e.g., solid tumor). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib described herein and/or the at least one anti-HER2 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or the at least one anti-HER2 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by mammography. In one embodiment, the size of a tumor derived from the cancer is measured by sonography. See Gruber et. al., 2013, BMC Cancer. 13:328.

In one embodiment of the methods or uses or product for uses provided described herein, response to treatment with tucatinib described herein and at least one anti-HER2 antibody described herein, promotes regression of a tumor derived from the cancer described herein (e.g., solid tumor). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein and/or anti-HER2 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or at least one anti-HER2 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by magnetic resonance imaging (MRI). In one embodiment, regression of a tumor is determined by computed tomography (CT). In one embodiment, regression of a tumor is determined by positron emission tomography (PET). In one embodiment, regression of a tumor is determined by mammography. In one embodiment, regression of a tumor is determined by sonography. See Gruber et. al., 2013, BMC Cancer. 13:328.

In one embodiment of the methods or uses or product for uses described herein, response to treatment with tucatinib described and at least one anti-HER2 antibody described herein is assessed by measuring the time of progression free survival after administration of tucatinib described herein and/or at least one anti-HER2 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or at least one anti-HER2 antibody described herein. In some embodiments, the subject exhibits progression-free survival of at least about 6 months after administration of tucatinib described herein and/or at least one anti-HER2 antibody described herein. In some embodiments, the subject exhibits progression-free survival of at least about one year after administration of tucatinib described herein and/or at least one anti-HER2 antibody described herein. In some embodiments, the subject exhibits progression-free survival of at least about two years after administration of tucatinib described herein and/or at least one anti-HER2 antibody described herein. In some embodiments, the subject exhibits progression-free survival of at least about three years after administration of tucatinib described herein and/or at least one anti-HER2 antibody described herein. In some embodiments, the subject exhibits progression-free survival of at least about four years after administration of tucatinib described herein and/or at least one anti-HER2 antibody described herein. In some embodiments, the subject exhibits progression-free survival of at least about five years after administration of tucatinib described herein and/or at least one anti-HER2 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or at least one anti-HER2 antibody described herein. In some embodiments, the subject exhibits progression-free survival of at least 6 months after administration of tucatinib described herein and/or at least one anti-HER2 antibody described herein. In some embodiments, the subject exhibits progression-free survival of at least one year after administration of tucatinib described herein and/or at least one anti-HER2 antibody described herein. In some embodiments, the subject exhibits progression-free survival of at least two years after administration of tucatinib described herein and/or at least one anti-HER2 antibody described herein. In some embodiments, the subject exhibits progression-free survival of at least three years after administration of tucatinib described herein and/or at least one anti-HER2 antibody described herein. In some embodiments, the subject exhibits progression-free survival of at least four years after administration of tucatinib described herein and/or at least one anti-HER2 antibody described herein. In some embodiments, the subject exhibits progression-free survival of at least five years after administration of tucatinib described herein and/or at least one anti-HER2 antibody described herein.

In one embodiment of the methods or uses or product for uses described herein, response to treatment with tucatinib described herein and at least one anti-HER2 antibody described herein is assessed by measuring the time of overall survival after administration of tucatinib described herein and/or at least one anti-HER2 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or at least one anti-HER2 antibody described herein. In some embodiments, the subject exhibits overall survival of at least about 6 months after administration of tucatinib described herein and/or at least one anti-HER2 antibody described herein. In some embodiments, the subject exhibits overall survival of at least about one year after administration of tucatinib described herein and/or at least one anti-HER2 antibody described herein. In some embodiments, the subject exhibits overall survival of at least about two years after administration of tucatinib described herein and/or at least one anti-HER2 antibody described herein. In some embodiments, the subject exhibits overall survival of at least about three years after administration of tucatinib described herein and/or at least one anti-HER2 antibody described herein. In some embodiments, the subject exhibits overall survival of at least about four years after administration of tucatinib described herein and/or at least one anti-HER2 antibody described herein. In some embodiments, the subject exhibits overall survival of at least about five years after administration of tucatinib described herein and/or at least one anti-HER2 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of tucatinib described herein and/or at least one anti-HER2 antibody described herein. In some embodiments, the subject exhibits overall survival of at least 6 months after administration of tucatinib described herein and/or at least one anti-HER2 antibody described herein. In some embodiments, the subject exhibits overall survival of at least one year after administration of tucatinib described herein and/or at least one anti-HER2 antibody described herein. In some embodiments, the subject exhibits overall survival of at least two years after administration of tucatinib described herein and/or at least one anti-HER2 antibody described herein. In some embodiments, the subject exhibits overall survival of at least three years after administration of tucatinib described herein and/or at least one anti-HER2 antibody described herein. In some embodiments, the subject exhibits overall survival of at least four years after administration of tucatinib described herein and/or at least one anti-HER2 antibody described herein. In some embodiments, the subject exhibits overall survival of at least five years after administration of tucatinib described herein and/or at least one anti-HER2 antibody described herein.

In one embodiment of the methods or uses or product for uses described herein, response to treatment with tucatinib described herein and at least one anti-HER2 antibody described herein is assessed by measuring the duration of response to tucatinib described herein and at least one anti-HER2 antibody described herein after administration of tucatinib described herein and/or anti-HER2 antibody described herein. In some embodiments, the duration of response to tucatinib described herein and at least one anti-HER2 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or at least one anti-HER2 antibody described herein. In some embodiments, the duration of response to tucatinib described herein and at least one anti-HER2 antibody described herein is at least about 6 months after administration of tucatinib described herein and/or at least one anti-HER2 antibody described herein. In some embodiments, the duration of response to tucatinib described herein and at least one anti-HER2 antibody described herein is at least about one year after administration of tucatinib described herein and/or at least one anti-HER2 antibody described herein. In some embodiments, the duration of response to tucatinib described herein and at least one anti-HER2 antibody described herein is at least about two years after administration of tucatinib described herein and/or at least one anti-HER2 antibody described herein. In some embodiments, the duration of response to tucatinib described herein and at least one anti-HER2 antibody described herein is at least about three years after administration of tucatinib described herein and/or at least one anti-HER2 antibody described herein. In some embodiments, the duration of response to tucatinib described herein and at least one anti-HER2 antibody described herein is at least about four years after administration of tucatinib described herein and/or at least one anti-HER2 antibody described herein. In some embodiments, the duration of response to tucatinib described herein and at least one anti-HER2 antibody described herein is at least about five years after administration of tucatinib described herein and/or at least one anti-HER2 antibody described herein. In some embodiments, the duration of response to tucatinib described herein and at least one anti-HER2 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or at least one anti-HER2 antibody described herein. In some embodiments, the duration of response to tucatinib described herein and at least one anti-HER2 antibody described herein is at least 6 months after administration of tucatinib described herein and/or at least one anti-HER2 antibody described herein. In some embodiments, the duration of response to tucatinib described herein and at least one anti-HER2 antibody described herein is at least one year after administration of tucatinib described herein and/or at least one anti-HER2 antibody described herein. In some embodiments, the duration of response to tucatinib described herein and at least one anti-HER2 antibody described herein is at least two years after administration of tucatinib described herein and/or at least one anti-HER2 antibody described herein. In some embodiments, the duration of response to tucatinib described herein and at least one anti-HER2 antibody described herein is at least three years after administration of tucatinib described herein and/or at least one anti-HER2 antibody described herein. In some embodiments, the duration of response to tucatinib described herein and at least one anti-HER2 antibody described herein is at least four years after administration of tucatinib described herein and/or at least one anti-HER2 antibody described herein. In some embodiments, the duration of response to tucatinib described herein and at least one anti-HER2 antibody described herein is at least five years after administration of tucatinib described herein and/or at least one anti-HER2 antibody described herein.

F. Compositions

In another aspect, the present invention provides a pharmaceutical composition comprising tucatinib described herein and a pharmaceutically acceptable carrier. In another aspect, the present invention provides a pharmaceutical composition comprising at least one anti-HER2 antibody described herein and a pharmaceutically acceptable carrier. In another aspect, the present invention provides a pharmaceutical composition comprising tucatinib described herein, at least one anti-HER2 antibody described herein, and a pharmaceutically acceptable carrier. In some embodiments, the at least one anti-HER2 antibody is a member selected from the group consisting of trastuzumab, pertuzumab, ado-trastuzumab emtansine, margetuximab, and a combination thereof. In some instances, the at least one anti-HER2 antibody is a combination of trastuzumab and pertuzumab. In some embodiments, the at least one anti-HER2 antibody is trastuzumab.

In some embodiments, tucatinib described herein is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib described herein is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, tucatinib described herein is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib described herein is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4.400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,4000, 9,500, 9,600, 9,700, 9,800, 9,900, 10,000, or more nM).

In some embodiments, the at least one anti-HER2 antibody described herein is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the at least one anti-HER2 antibody described herein is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the at least one anti-HER2 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the at least one anti-HER2 antibody is present at a concentration of at least about 1,000 nM to 10,000 nM (e.g., at least about 1,000, 1,100, 1,200, 1,300, 1,400, 1,500, 1,600, 1,700, 1.800, 1,900, 2,000, 2,100, 2,200, 2,300, 2,400, 2.500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, there is a pharmaceutical composition comprising at least one anti-HER2 antibody described herein, wherein the at least one anti-HER2 antibody comprises a first anti-HER2 antibody and a second anti-HER2 antibody, wherein each of the first anti-HER2 antibody and the second anti-HER2 antibody are present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, each of the first anti-HER2 antibody and the second anti-HER2 antibody described herein are present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, each of the first anti-HER2 antibody and the second anti-HER2 antibody are present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, each of the first anti-HER2 antibody and the second anti-HER2 antibody are present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM). In some embodiments, the pharmaceutical composition is for subcutaneous administration. In some embodiments, the pharmaceutical composition comprises hyaluronidase.

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a combination of drugs (e.g., tucatinib described herein and at least one anti-HER2 antibody described herein), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib described herein, at least one anti-HER2 antibody described herein, or a combination thereof) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein (e.g., tucatinib described herein, at least one anti-HER2 antibody described herein, or a combination thereof), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib described herein, at least one anti-HER2 antibody described herein, or a combination thereof) and the powder of a suitable carrier or excipient. The compositions for systemic administration can be represented by, but not limited to, tablets, capsules, pills, syrups, solutions, and suspensions.

In some embodiments, the compositions (e.g., tucatinib described herein, at least one anti-HER2 antibody described herein, or a combination thereof) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib described herein, at least one anti-HER2 antibody described herein, or a combination thereof) can be made as implants, oily injections, or as particulate systems. For a broad overview of delivery systems see Banga, A. J., Therapeutic Peptides and Proteins: Formulation, Processing, and Delivery Systems, Technomic Publishing Company, Inc., Lancaster, PA, (1995), which is incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, PA (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670: 5,413,797; 5,268,164: 5,004,697; 4,902,505: 5,506,206, 5,271,961; 5,254,342 and 5,534,496, each of which is incorporated herein by reference.

For oral administration of a combination of tucatinib described herein and/or at least one anti-HER2 antibody described herein, a pharmaceutical composition or a medicament can take the form of, for example, a tablet or a capsule prepared by conventional means with a pharmaceutically acceptable excipient. The present invention provides tablets and gelatin capsules comprising tucatinib described herein, at least one anti-HER2 antibody described herein, or a combination thereof, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol: for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib described herein, at least one anti-HER2 antibody described herein, or a combination thereof include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib described herein, at least one anti-HER2 antibody described herein, or a combination thereof) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib described herein, at least one anti-HER2 antibody described herein, or a combination thereof) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib described herein, at least one anti-HER2 antibody described herein, or a combination thereof) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

G. Articles of Manufacture and Kits

In another aspect, the present invention provides an article of manufacture or kit for treating or ameliorating the effects of breast cancer in a subject, the article of manufacture or kit comprising a pharmaceutical composition of the present invention (e.g., a pharmaceutical composition comprising tucatinib described herein, at least one anti-HER2 antibody described herein, or a combination thereof). In some embodiments, the at least one anti-HER2 antibody is trastuzumab, pertuzumab, ado-trastuzumab emtansine, margetuximab, or a combination thereof. In some instances, the at least one anti-HER2 antibody is a combination of trastuzumab and pertuzumab. In some embodiments, the at least one anti-HER2 antibody is trastuzumab.

The articles of manufacture or kits are suitable for treating or ameliorating the effects of cancers, particularly solid tumors that have been determined to express a mutant form of HER2. In some embodiments, the cancer is an advanced cancer.

Materials and reagents to carry out the various methods of the present invention can be provided in articles of manufacture or kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Articles of manufacture or kits can contain chemical reagents as well as other components. In addition, the articles of manufacture or kits of the present invention can include, without limitation, instructions to the user, apparatus and reagents for administering combinations of tucatinib described herein and anti-HER2 antibodies described herein or pharmaceutical compositions thereof, sample tubes, holders, trays, racks, dishes, plates, solutions, buffers, or other chemical reagents. In some embodiments, the articles of manufacture or kits contain instructions, apparatus, or reagents for determining the genotype of a gene (e.g., KRAS, NRAS, BRAF) or determining the expression of HER2 in a sample. Articles of manufacture or kits of the present invention can also be packaged for convenient storage and safe shipping, for example, in a box having a lid.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase H Basket Study of Tucatinib in Combination with Trastuzumab in Subjects with Previously Treated, Locally-Advanced Unresectable or Metastatic Solid Tumors Driven by HER2 Alterations

This multi-cohort, open label, multicenter, international Phase 2 clinical study is designed to assess the activity, safety, and tolerability of tucatinib in combination with trastuzumab for the treatment of selected solid tumors with HER2 alterations. Subjects will be enrolled into separate cohorts based on tumor histology and HER2 alteration status.

There are 5 tumor specific cohorts with HER2 overexpression/amplification (cervical cancer [Cohort 1], uterine cancer [Cohort 2], biliary tract cancer [Cohort 3], urothelial cancer [Cohort 4], and non-squamous non-small cell lung cancer [NSCLC] [Cohort 5]), 2 tumor specific cohorts with HER2 mutations (non-squamous NSCLC [Cohort 7] and breast cancer [Cohort 8]), and 2 cohorts which will enroll all other HER2 amplified/overexpressed solid tumor types (except breast, gastric or gastroesophageal junction adenocarcinoma [GEC], and colorectal cancer [CRC]) or HER2-mutated solid tumor types (Cohorts 6 and 9 respectively).

If a sufficient number of subjects with a particular tumor type are enrolled in Cohorts 6 or 9, the sponsor may evaluate that tumor type in a separate cohort, drawn from optional Cohorts 10 to 15. If any optional cohort is opened, all subjects enrolled in Cohorts 6 or 9 with the applicable tumor type will be reassigned to the new tumor-specific cohort: these subjects will be replaced in Cohorts 6 and 9.

In Stage 1, up to approximately 12 response-evaluable subjects may be enrolled in each of Cohorts 1 to 5, and 7. If sufficient activity is observed in Stage I for a particular cohort, up to a total of 30 response-evaluable subjects will be enrolled in the cohort (Stage 2 expansion) to further characterize the activity and safety of the study regimen in the given disease and HER2 alteration type. Cohorts 6, 8, and 9 will enroll up to 30 response-evaluable subjects without undergoing the Stage 1 assessment in 12 subjects. Subjects who are not response-evaluable will be replaced. The following table shows the definition of cohorts:

### Definition of Cohorts According to Disease Type and HER2 Alterations, and Number of Subjects

| | HER2 alteration | | N subjects per cohort | |
|---|---|---|---|---|
| | Overexpression/ | | | Stage 1 and |
| | amplification | Mutations | Stage 1 | Stage 2 |
| Cervical cancer | Cohort 1 | | 12 | 30 |
| Uterine cancer | Cohort 2 | | 12 | 30 |
| Biliary tract cancer (gallbladder cancer and cholangiocarcinoma) | Cohort 3 | | 12 | 30 |
| Urothelial cancer | Cohort 4 | | 12 | 30 |
| Non-squamous NSCLC | Cohort 5 | Cohort 7 | 12 | 30 |
| Other solid tumors (except breast cancer, GEC, and CRC) | Cohort 6 | | 30 | |
| Breast cancer | | Cohort 8 | 30 | |
| All solid tumors (except breast and non-squamous NSCLC) | | Cohort 9 | 30 | |
| Optional tumor specific Cohorts | | | | |
| Cervical cancer | | Cohort 10 | 12 | 30 |
| Uterine cancer | | Cohort 11 | 12 | 30 |
| Biliary tract cancer | | Cohort 12 | 12 | 30 |
| Urothelial cancer | | Cohort 13 | 12 | 30 |
| Additional tumor specific | Cohort 14 | Cohort 15 | 12 | 30 |

Study treatment is composed of tucatinib 300 mg twice daily (BID) by oral administration (PO) combined with trastuzumab 8 mg/kg intravenously (IV) on Cycle 1 Day 1 and then 6 mg/kg every 21 days starting on Cycle 2 Day 1. Subjects with hormone receptor (HR) positive (HR+), HER2-mutated breast cancer will also receive, in combination with tucatinib and trastuzumab, fulvestrant 500 mg intramuscular (IM) once every 4 weeks starting from Cycle 1 Day 1, as well as on Cycle 1 Day 15. A Safety Monitoring Committee (SMC) will be responsible for monitoring the safety of subjects in the study at regular intervals. Subjects will continue study treatment until the occurrence of radiographic or clinical progression, unacceptable toxicity, withdrawal of consent, death, or study closure. Following treatment discontinuation, disease progression, further anti-cancer therapy, and survival status will be monitored until withdrawal of consent, death, or study closure. The study will be closed approximately 3 years after the last subject is enrolled or when no subjects remain in long-term follow-up, whichever occurs first. Additionally, the sponsor may terminate the study at any time.

### Study Population

Inclusion Criteria

All subjects must meet the following eligibility criteria for enrollment:
1. Histologically or cytologically confirmed diagnosis of locally-advanced unresectable or metastatic solid tumor, including primary brain tumors
2. Subjects with disease types other than breast cancer, biliary tract cancer, and non-squamous NSCLC: Disease progression on or after the most recent systemic therapy for locally-advanced unresectable or metastatic disease
3. Subjects with any breast cancer subtype:
   a. Must have HER2-mutated disease which does not display HER2 overexpression/amplification
   b. must have completed 21 prior line of treatment (chemotherapy, endocrine therapy, or targeted therapy) for locally-advanced unresectable or metastatic breast cancer
4. Subjects with biliary tract cancer: must have completed 21 prior line of treatment (chemotherapy, endocrine therapy, or targeted therapy)
5. Subjects with non-squamous NSCLC: has relapsed from or is refractory to standard treatment or for which no standard treatment is available
6. Disease demonstrating HER2 alterations (overexpression/amplification or HER2 activating mutations), as determined by local or central testing processed in a Clinical Laboratory Improvement Amendments (CLIA)- or International Organization for Standardization (ISO)-accredited laboratory, according to one of the following:
   a. HER2 overexpression/amplification from fresh or archival tumor tissue or blood utilizing one of the following tests, in subjects with tumor types other than breast cancer, GEC, or CRC:
      i. HER2 overexpression (3+ immunohistochemistry IHC) (breast or gastric algorithms)
      ii. HER2 amplification by in situ hybridization assay (fluorescence in situ hybridization [FISH] or chromogenic in situ hybridization signal ratio ≥2.0 or gene copy number >6)
      iii. HER2 amplification in tissue by next generation sequencing (NGS) assay
      iv. HER2 amplification in circulating tumor DNA (ctDNA) by blood-based NGS assay
   b. Known activating HER2 mutations detected in fresh or archival tumor tissue or blood by NGS assay, including:
      Extracellular domain: G309A/E; S310F/Y; C311R/S; C334S
      Kinase domain: T733I; L755P/S: 1767M: L768S; D769N/Y/H; Y772; A775; G776; V777L/M; G778: T798: L841V. V8421; N857S, T862A, L869R, H878Y, R896C
      Transmembrane/juxtamembrane domain: S653C, 1655V; V659E; G660D; R678Q; V697.
      Other mutations not listed may be eligible with approval by the medical monitor.
7. Have measurable disease per RECIST v1.1 criteria according to investigator assessment
8. Be at least 18 years of age at time of consent, or considered an adult by local regulations
9. Have Eastern Cooperative Oncology Group (ECOG) performance status 0 or 1
10. Have a life expectancy of at least 3 months, in the opinion of the investigator
11. Have adequate hepatic function as defined by the following:
   a. Aspartate aminotransferase (AST) and alanine aminotransferase (ALT)≤3× upper limit of normal (ULN) (≤5×ULN if liver metastases are present)
   b. Total bilirubin ≤1.5×ULN. Exception: subjects with known history of Gilbert's Syndrome and normal direct bilirubin, AST, and ALT are eligible
12. Have adequate baseline hematologic parameters as defined by:
   a. Absolute neutrophil count (ANC) ≥1.0×109/L
   b. Platelet count ≥100×109/L: subjects with stable platelet count from 75 to 100×109/L may be included with approval from Medical Monitor
   c. Hemoglobin ≥9.0 g/dL; subjects with hemoglobin ≥8 and <9 g/dL may be included with approval from the Medical Monitor
   d. In subjects transfused before study entry, transfusion must be ≥14 days prior to start of therapy to establish adequate hematologic parameters independent from transfusion support
13. Estimated glomerular filtration rate (GFR) ≥30 mL/min/1.73 m² using the Modification of Diet in Renal Disease (MDRD) study equation
14. International normalized ratio (INR) and activated partial thromboplastin time (aPTT) ≤1.5×ULN unless receiving a medication known to alter INR and aPTT
15. Left ventricular ejection fraction (LVEF) ≥50% as assessed by echocardiogram or multiple-gated acquisition scan (MUGA) documented within ≤28 days prior to first dose of study treatment
16. For subjects of childbearing potential, the following stipulations apply:
   a. Must have a negative serum pregnancy test (minimum sensitivity of 25 mIU/mL or equivalent units of beta human chorionic gonadotropin [β-hCG]) result within 7 days prior to the first dose of study treatment. A subject with a false positive result and documented verification that the subject is not pregnant is eligible for participation.
   b. Must agree not to try to become pregnant during the study and for at least 7 months after the final dose of any study drug, and, if applicable, for at least 2 years after the final dose of fulvestrant.
   c. Must agree not to breastfeed or donate ova, starting at time of informed consent and continuing through at least 7 months after the final dose of any study drug, and, if applicable, at least 2 years after the final dose of fulvestrant.
   d. If sexually active in a way that could lead to pregnancy, must consistently use 2 highly effective methods of birth control starting at the time of informed consent and continuing throughout the study and for at least 7 months after the final dose of any study drug, and, if applicable, for at least 2 years after the final dose of fulvestrant.
17. For subjects who can father children, the following stipulations apply:
   a. Must agree not to donate sperm starting at time of informed consent and continuing throughout the study period and for at least 7 months after the final dose of any study drug, and, if applicable, for at least 2 years after the final dose of fulvestrant.
   b. If sexually active with a person of childbearing potential in a way that could lead to pregnancy, must consistently use 2 highly effective methods of birth control, starting at time of informed consent and continuing throughout the study and for at least 7 months after the final dose of any study drug, and, if applicable, for at least 2 years after the final dose of fulvestrant.
   c. If sexually active with a person who is pregnant or breastfeeding, must consistently use one of 2 methods of birth control starting at time of informed consent and continuing throughout the study and for at least 7 months after the final dose of any study drug, and, if applicable, for at least 2 years after the final dose of fulvestrant.
18. Subject must provide signed informed consent that has been approved by an institutional review board/independent ethics committee (IRB/IEC) prior to initiation of any study-related tests or procedures that are not part of standard-of-care for the subject's disease
19. Subject must be willing and able to comply with study procedures

Exclusion Criteria
1. Subjects with breast cancer, GEC, or CRC whose disease shows HER2 amplification/overexpression.
2. Previous treatment with HER2-directed therapy; subjects with uterine serous carcinoma may have received prior trastuzumab
3. Known hypersensitivity to any component of the drug formulation of tucatinib or trastuzumab (drug substance, excipients, murine proteins), or any component of the drug formulation of fulvestrant in subjects with HR+HER2-mutated breast cancer
4. History of exposure to a 360 mg/m² doxorubicin-equivalent cumulative dose of anthracyclines
5. Treatment with any systemic anti-cancer therapy, radiation therapy, or experimental agent within ≤3 weeks of first dose of study treatment or are currently participating in another interventional clinical trial.
6. Have any toxicity related to prior cancer therapies that has not resolved to ≤Grade 1, with the following exceptions:
   a. Alopecia
   b. Congestive heart failure (CHF), which must have been ≤Grade 1 in severity at the time of occurrence, and must have resolved completely
   c. Anemia, which must have resolved to ≤Grade 2
7. Have clinically significant cardiopulmonary disease such as:
   a. Ventricular arrhythmia requiring therapy
   b. Symptomatic hypertension or uncontrolled hypertension as determined by investigator
   c. Any history of symptomatic CHF
   d. Severe dyspnea at rest (National Cancer Institute Common Terminology Criteria for Adverse Events [NCI CTCAE] Grade 3 or above) due to complications of advanced malignancy
   e. Hypoxia requiring supplementary oxygen therapy except when oxygen therapy is needed only for obstructive sleep apnea
8. Have known myocardial infarction or unstable angina within 6 months prior to first dose of study treatment
9. Known to be positive for hepatitis B by surface antigen expression. Known to be positive for hepatitis C infection (positive by polymerase chain reaction). Subjects who have been treated for hepatitis C infection are permitted if they have documented sustained virologic response of 12 weeks
10. Presence of known chronic liver disease
11. Subjects known to be positive for human immunodeficiency virus (HIV) are excluded if they meet any of the following criteria:
   CD4+ T-cell count of <350 cells/μL
   Detectable HIV viral load
   History of an opportunistic infection within the past 12 months
   On stable antiretroviral therapy for <4 weeks
12. Are pregnant, breastfeeding, or planning a pregnancy from time of informed consent until 7 months after the final dose of any study drug, and, if applicable, for at least 2 years after the final dose of fulvestrant
13. Have inability to swallow pills
14. Have used a strong cytochrome P450 (CYP) 2C8 inhibitor within 5 half-lives of the inhibitor, or have used a strong CYP3A4 or CYP2C8 inducer within 5 days prior to start of treatment
15. Have any other medical, social, or psychosocial factors that, in the opinion of the investigator, could impact safety or compliance with study procedures
16. History of another malignancy within 2 years prior to screening, with the exception of those with a negligible risk of metastasis or death (e.g., 5-year OS of ≥90%), such as adequately treated carcinoma in situ of the cervix, non-melanoma skin carcinoma, localized prostate cancer, ductal carcinoma in situ, or Stage I uterine cancer
17. Subjects with known central nervous system (CNS) lesions must not have any of the following:
   a. Any untreated brain lesions >2.0 cm in size, unless approved by the medical monitor
   b. Ongoing use of systemic corticosteroids for control of symptoms of brain lesions at a total daily dose of >2 mg of dexamethasone (or equivalent). However, subjects on a chronic stable dose of ≤2 mg total daily of dexamethasone (or equivalent) may be eligible, following approval by the medical monitor
   c. Any brain lesion thought to require immediate local therapy, including (but not limited to) a lesion in an anatomic site where increase in size or possible treatment-related edema may pose risk to subject (e.g., brain stem lesions). Subjects who undergo local treatment for such lesions identified by screening brain magnetic resonance imaging (MRI) may still be eligible for the study based on criteria described under CNS inclusion criteria b
   d. Known or suspected leptomeningeal disease as documented by the investigator
   e. Have poorly controlled (>1/week) generalized or complex partial seizures, or manifest neurologic progression due to brain lesions notwithstanding CNS-directed therapy

### Number of Planned Subjects

Approximately 162 to 270 subjects may be enrolled in the study. This is comprised of up to approximately 12 to 30 subjects in each of Cohorts 1 to 5 and Cohort 7, and up to approximately 30 subjects in each of Cohorts 6, 8, and 9. Additional subjects may be enrolled if any of the optional Cohorts 10 to 15 are opened. Subjects initially enrolled in Cohorts 6 or 9 who are reassigned to an optional cohort will be replaced.

### Investigational Product, Dose, and Mode of Administration

Subjects will receive combination therapy of the investigational medicinal products tucatinib and trastuzumab. Study treatment will be given on a 21-day cycle, with tucatinib every day and trastuzumab on Day 1. Tucatinib 300 mg will be administered orally (PO) twice daily (BID) continuously starting from Cycle 1 Day 1 onwards. Trastuzumab 8 mg/kg will be administered IV on Cycle 1 Day 1 and then will be administered at 6 mg/kg every 21 days starting on Cycle 2 Day 1. However, if trastuzumab IV was administered within the 4 weeks prior to treatment initiation, trastuzumab 6 mg/kg IV should be administered on Cycle 1 Day 1. Fulvestrant 500 mg will be administered intramuscular (IM) once every 4 weeks starting from Cycle 1 Day 1, as well as on Cycle 1 Day 15. Cycles are defined by trastuzumab administration, with a new cycle starting whenever the Day 1 infusion of trastuzumab is administered. If trastuzumab is discontinued, cycles will be defined as occurring every 21 days from the last Day 1 administration of trastuzumab.

### Treatment Schedule

| Agent | Dose | | Route | Period | Daily frequency | Dosing Schedule |
|---|---|---|---|---|---|---|
| Tucatinib | 300 | mg | PO | All cycles | BID | Every day, from Cycle 1 Day 1 |
| Trastuzumab | 8 | mg/kg | IV | Cycle 1 | Once | Day 1 |
| | 6 | mg/kg | IV | Cycles >1 | Once | Day 1 |
| Fulvestrant | 500 | mg | IM | Every 4 weeks | Once | Starting from Cycle 1 Day 1 |
| (if applicable) | | | | Cycle 1 | Once | Day 15 |

### Duration of Treatment

Study treatment will continue until unacceptable toxicity, occurrence of radiographic progression or clinical progression, withdrawal of consent, death, or study closure. If a study drug (tucatinib, trastuzumab, or fulvestrant) is discontinued, study treatment can continue with remaining study drug(s).

### Efficacy Assessment

Disease response will be assessed by the investigator according to RECIST v1.1. Treatment decisions will be made based upon local assessment of radiologic scans. Radiographic disease assessments will evaluate all known sites of disease, preferably using high quality spiral contrast computed tomography (CT) (with oral and/or IV contrast), and covering, at a minimum, the chest, abdomen, and pelvis. Positron emission tomography-CT scans (if high quality CT scan is included) and/or MRI scans may also be used as appropriate, as well as additional imaging of any other known sites of disease. In subjects with breast or lung cancer, a contrast MRI scan of the brain should be performed at screening. Subjects with known or suspected brain lesions should undergo brain MRIs during treatment and follow-up according to the same assessment schedule as for other disease assessments. If contrast is contraindicated (i.e., in subjects with contrast allergy or impaired renal clearance), a non-contrast CT scan of the chest may be performed instead, with MRI scans of the abdomen and pelvis. For each subject, the same imaging modality as used at screening/baseline should be used throughout the study, unless otherwise clinically indicated. Images will be collected by an independent central review (ICR) facility for possible future analysis. Disease assessments will be done at screening/baseline, and every 6 weeks for first 24 weeks then every 12 weeks, irrespective of dose interruptions.

Responses complete response or partial response (CR or PR) will be confirmed with repeat scans at least 4 weeks after first documentation of response. The schedule for response assessments should not be adjusted after the confirmatory scan (e.g., CR at Week 6, confirmatory scans at Week 10-12, next assessment due at Week 12). Tumor imaging should also be performed whenever disease progression is suspected.

Subjects will be considered evaluable for response if they (1) had a baseline disease assessment, (2) received study treatment, and (3) had a post-baseline disease assessment or discontinued treatment due to documented disease progression or clinical progression.

Subjects that discontinue study treatment for reasons other than documented progressive disease or death will continue to have disease assessments every 6 weeks (±1 week) until 24 weeks after treatment initiation, then every 12 weeks (±1 week), until the occurrence of documented disease progression per RECIST v1.1, death, withdrawal of consent, lost to follow-up, or study closure.

Follow-up for survival and subsequent anti-cancer therapy will occur approximately every 12 weeks and continue until death, withdrawal of consent, lost to follow-up, or study closure.

### Pharmacokinetic Assessments

Blood samples for PK assessment of trough tucatinib drug levels will be collected in all subjects on Day 1 of Cycles 3 to 6, prior to administration of tucatinib. On Day 1 of Cycle 3, PK assessments of peak levels of tucatinib will be performed 1 to 4 hours after administration of tucatinib. Plasma concentrations of tucatinib will be determined using validated liquid chromatography (LC)-mass spectrometry (MS)/MS methods. PK parameters will be summarized using descriptive statistics.

### HER2 Testing For Eligibility and Biomarker Assessments

Study eligibility requirements for HER2 overexpressing/amplified disease and HER2-mutated disease are to be met by assays performed pre-study (assessments undertaken prior to any study-related actives) or in pre-screening, as follows:
1. Previously established HER2 alterations: HER2 eligibility can be demonstrated via HER2 overexpression or amplification in an IHC/ISH assay of tumor tissue or HER2 amplification or activating mutations in an NGS assay of ctDNA or tumor tissue, processed locally in a CLIA- or ISO accredited laboratory before enrollment in the study.
2. Pre-screening for HER2 alterations: if HER2 alterations have not been detected in pre-study assessments, HER2 eligibility may alternatively be established during pre-screening, up to 3 months prior to the Screening visit, via a next generation sequencing (NGS) assay of ctDNA evaluating the presence of HER2 amplification or mutations.
3. Additional samples for exploratory analyses: For exploratory analysis, all subjects will provide a blood sample for NGS assay of ctDNA and archival tumor tissue or a fresh tumor biopsy, if available, either in pre-screening or at the Screening visit, if pre-screening did not occur. However, the blood sample does not need to be drawn, if previously obtained by the sponsor, since the end of prior therapy. The results of this additional testing will not be used to determine eligibility.

Archival tumor tissue samples should be the most recent tissue sample available. If an archival sample is not available, a fresh biopsy will be undertaken at pre-screening or the Screening visit, if the subject has an available tumor lesion and consents to the biopsy. Subjects with no archival tissue and whose tumors are considered not accessible or appropriate for biopsy are eligible for enrollment, following approval by the medical monitor.

Additional biomarker assessments may include an exploratory assessment of HER2 mutations or other mutations as potential biomarkers of response. Additional exploratory analyses including but not limited to IHC and NGS analysis may be performed to interrogate biomarkers that are associated with tumor growth, survival, and resistance to targeted therapeutics. This assessment may enable the correlation of additional biomarkers with treatment outcome and may ultimately guide or refine patient selection strategies to better match tucatinib regimens with tumor phenotype/genotype in the future.

### Safety Assessment

Safety assessments will include the surveillance and recording of adverse events (AEs), including serious adverse events (SAEs) and adverse events of special interest (AESI), physical examination findings, vital signs, 12-lead electrocardiograms, concomitant medications, pregnancy testing, and laboratory tests. Assessment of cardiac ejection fraction will be performed using MUGA scan or echocardiogram. An ongoing, real-time review of subject safety and SAEs will be conducted by the sponsor's Drug Safety Department. The SMC will be responsible for monitoring the safety of subjects in the study at regular intervals. AE and laboratory abnormality severity will be graded using the National Cancer Institute Common Terminology Criteria for Adverse Events (NCI CTCAE), version 5.

### Statistical Methods

Safety and efficacy will be assessed using descriptive statistics, including the number of observations, mean, median, standard deviation, minimum and maximum for continuous variables, and the number and percentages (of non-missing) per category for categorical variables. Confirmed objective response rate (ORR) per investigator is defined as the proportion of subjects with confirmed CR or PR, per RECIST v1.1. The 2-sided 90% exact confidence interval (CI) using Clopper-Pearson method will be calculated for the response rates.

The primary analysis of the study will be performed when 30 response-evaluable subjects in each cohort have been followed for at least 12 weeks or have documented disease progression. The primary efficacy endpoint of confirmed ORR per RECIST v1.1 will be estimated for each cohort based on the response-evaluable set, comprising all subjects who received any amount of study treatment who are considered evaluable for response. The 90% exact CIs using the Clopper-Pearson method will be provided. The confirmed ORR may also be summarized combining cohorts with same disease type.

Interim futility analyses will be performed separately for Cohorts 1 to 5 and 7 after approximately 12 subjects of a given cohort (Stage 1) have been treated and had at least two response assessments post-baseline or had disease progression.

The Bayesian predictive probability approach will be used to determine the futility criteria. At the time of each interim analysis, the predictive probability of success (PPoS) will be calculated. A PPoS <20% indicates that it is unlikely the ORR will be better than the response rate of current standard of care at the end of the study given the interim result. Based on activity and safety data, together with the PPoS, a cohort may be stopped early by the sponsor. Cohorts that successfully pass the interim analysis for futility may, at the sponsor's decision, continue to enroll up to an additional 18 response-evaluable subjects, totaling up to 30 response-evaluable subjects for each tumor cohort. A cohort may be expanded to Stage 2 earlier if the futility rule is cleared before 12 subjects, in other words, if the minimal required responses are observed in fewer than 12 subjects.

Safety measurements will be summarized by descriptive statistics based on the safety analysis set. The safety analysis set will include all subjects who received any amount of study treatment.

For those cohorts that are expanded to Stage 2, a total of 30 subjects (combining two stages) will be treated in each cohort. For a sample size of 30 subjects, assuming confirmed ORR is between 10% and 30%, the 2-sided 90% exact CIs are summarized below:

| Confirmed ORR | 90% Exact CI (N = 30) |
|---|---|
| 10% | (3%, 24%) |
| 20% | (9%, 36%) |
| 30% | (17%, 47%) |

### Investigational Products

Tucatinib drug product is supplied as both a coated yellow oval-shaped tablet in a 150 mg dosage strength and a coated yellow round convex tablet in a 50 mg dosage strength. The tablets are manufactured from a drug product intermediate amorphous dispersion of tucatinib in polyvinylpyrrolidone-vinyl acetate copolymer, which is then combined with the pharmaceutical excipients (microcrystalline cellulose, sodium chloride, potassium chloride, sodium bicarbonate, silicon dioxide, crospovidone, and magnesium stearate), and compressed into tablets.

Trastuzumab is a humanized immunoglobulin G1 (IgG1) kappa monoclonal antibody which binds to the extracellular domain of HER2; it mediates antibody-dependent cellular cytotoxicity by inhibiting proliferation of cells which over express the HER2 protein. Trastuzumab is indicated for adjuvant treatment of HER2-overexpressing node positive or node negative breast cancer, in combination with paclitaxel for first-line treatment of HER2-overexpressing mBC, as a single agent for treatment of HER2-overexpressing breast cancer in patients who have received one or more chemotherapy regimens for metastatic disease, and in combination with cisplatin and capecitabine or 5-fluorouracil, for the treatment of patients with HER2-overexpressing metastatic GEC who have not received prior treatment for metastatic disease.

Single-dose vial (150 mg/vial) as a lyophilized sterile powder for reconstitution is commercially available and should be prepared and administered per instructions in the trastuzumab package insert for administration instructions. Trastuzumab will be administered IV under the direction of the investigator.

Fulvestrant is an ER antagonist approved for use in treatment of HR+mBC in postmenopausal women with disease progression following antiestrogen therapy.

Administration of fulvestrant will be required in patients with HR+HER2-mutant breast cancer.

### Dose Modifications

### Tucatinib

Up to 3 dose reductions of tucatinib are allowed. Subjects who would require a dose reduction to below 150 mg BID should discontinue treatment with tucatinib. Dose reductions of larger intervals than those described in may be made at the discretion of the investigator, with approval by the medical monitor.

### Tucatinib—Recommended Dose Reduction Schedule for Adverse Events

| Dose Reduction Schedule | Tucatinib Dose Level |
|---|---|
| Starting dose | 300 mg PO BID ^{a} |
| 1st dose reduction | 250 mg PO BID |
| 2nd dose reduction | 200 mg PO BID |
| 3rd dose reduction | 150 mg PO BID |
| Requirement for further dose reduction | Discontinue tucatinib |

^{a} Dose reductions of greater intervals than those recommended in this table (i.e., more than 50 mg per dose reduction) may be made if considered clinically appropriate by the investigator and approved by the medical monitor. However, tucatinib may not be dose reduced below 150 mg BID.

### Trastuzumab

In the event of Grade ≥3 trastuzumab-related AEs, hold trastuzumab until the AE has resolved to Grade ≤1 or pretreatment levels. Resume trastuzumab at the same dose; the trastuzumab dose may not be reduced. If the Day 1 dosing of trastuzumab is delayed by >1 week, the IV loading dose of 8 mg/kg should be given per approved dosing instructions. If dosing of trastuzumab is held for >3 weeks, medical monitor approval is required before restarting trastuzumab.

### Fulvestrant

For subjects who develop moderate hepatic impairment (Child-Pugh class B) on study, a discussion with the medical monitor is required. If the subject is approved by the medical monitor to continue on study, a dose reduction to fulvestrant 250 mg is required.

### Objectives

This study will evaluate the efficacy, safety, and pharmacokinetics (PK) of tucatinib in combination with trastuzumab in subjects with solid tumors displaying HER2 alterations. Specific objectives and corresponding endpoints for the study are summarized below:

### Objectives and Corresponding Endpoints

| Primary Objective | Corresponding Endpoints: |
|---|---|
| To evaluate the antitumor activity of | Primary endpoint: |
| tucatinib given in combination with | Confirmed objective response rate (ORR; confirmed complete |
| trastuzumab in subjects with previously | response [CR] or partial response [PR]) according to Response |
| treated, locally-advanced unresectable or | Evaluation Criteria in Solid Tumors (RECIST) v1.1 per |
| metastatic HER2 overexpressing/amplified | investigator assessment |
| or mutated solid tumors | Secondary endpoints: |
| | Disease control rate (DCR; confirmed CR or PR, or stable |
| | disease) per investigator assessment |
| | Duration of response (DOR; confirmed CR or PR) per |
| | investigator assessment |
| | Progression-free survival (PFS) per investigator assessment |
| | Overall survival (OS) |
| Secondary Objective | Corresponding Endpoints |
| To evaluate the safety and tolerability of | Type, incidence, severity, seriousness, and relatedness of |
| tucatinib given in combination with | adverse events (AEs) |
| trastuzumab with or without fulvestrant | Type, incidence, and severity of laboratory abnormalities |
| | Frequency of treatment interruptions, dose reductions, and |
| | treatment discontinuations due to AEs |
| | Other relevant safety variables including AEs of special |
| | interest (AESIs) |
| Exploratory Objectives | Corresponding Endpoints |
| To evaluate the pharmacokinetics (PK) | Plasma concentrations of tucatinib |
| of tucatinib | |
| To explore any correlations between | Potential biomarkers of response, resistance, or toxicity may |
| tissue and blood-based biomarkers and | be evaluated in blood and/or tumor tissue |
| clinical outcomes | |
| To evaluate patient-reported outcomes | Change from baseline in health-related quality of life |
| (PROs) | (HRQoL), as assessed by the European Quality of Life 5- |
| | Dimension 5-Level (EQ-5D-5L) |

### Discontinuation of Study Treatment

A subject's study treatment may be discontinued for any of the following reasons:
Progressive disease
AE
Pregnancy or begins breastfeeding while on study
Investigator decision, due to clinical progression
Investigator decision, other
Subject decision, non-AE
Note: Ensure that subjects who decide to stop treatment because of an AE are not included in this rationale.
Study termination by sponsor
Other, non-AE

If a study drug (tucatinib, trastuzumab, or fulvestrant) is discontinued, study treatment can continue with remaining study drug(s). Subjects who discontinue study treatment for reasons other than documented progressive disease or death will continue to have disease assessments every 6 weeks (±1 week) until 24 weeks after treatment initiation, then every 12 weeks (±1 week), until the occurrence of disease progression per RECIST v1.1, death, withdrawal of consent, lost to follow-up, or study closure.

Follow-up for survival and subsequent anti-cancer therapy will occur approximately every 12 weeks and continue until death, withdrawal of consent, lost to follow-up, or study closure.

Every effort should be made to confirm disease progression (per RECIST 1.1) whenever possible. However, in instances where patients appear to have progressive symptoms for whom it is not possible or feasible to undergo radiologic assessment, investigators may remove the patient from study treatment due to “physician decision due to clinical progression.” Use of this reason for removing such patients from study treatment should be restricted to those cases in which it is not clinically appropriate for the patient to undergo further radiologic assessment and where there is clinical confidence for cancer progression in the absence of radiographic confirmation. Special consideration should be given to ensure that other possible reasons, particularly AEs, are not a more accurate description of the reason for study drug discontinuation in these cases.

Subject Withdrawal from Study

Any subject may be discontinued from the study for any of the following reasons:
Subject withdrawal of consent
Study termination by sponsor
Lost to follow-up
Death
Other

### Example 2: Treatment with Tucatinib Increases Overall and Plasma Membrane-Bound HER2 Levels

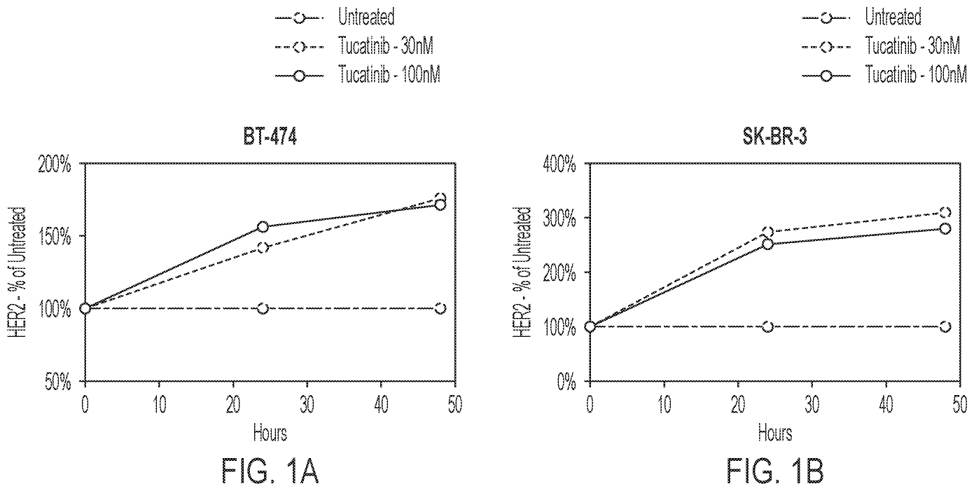

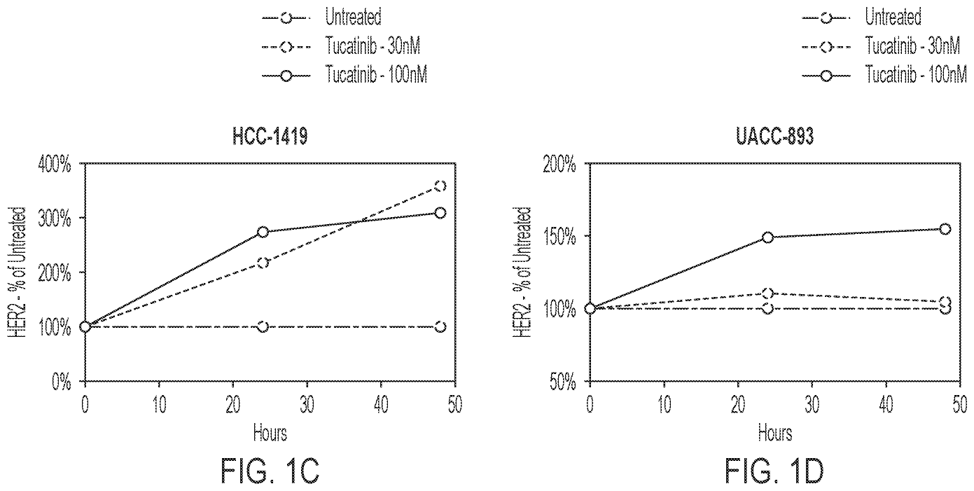

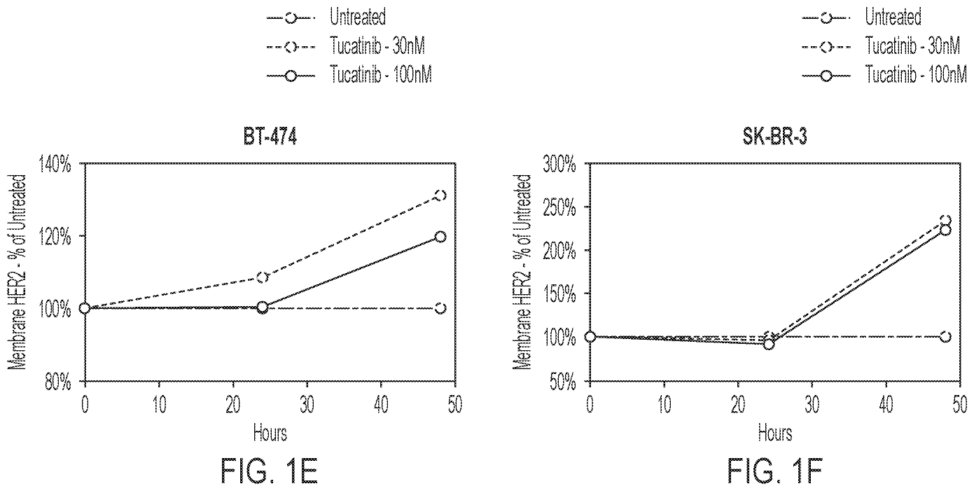

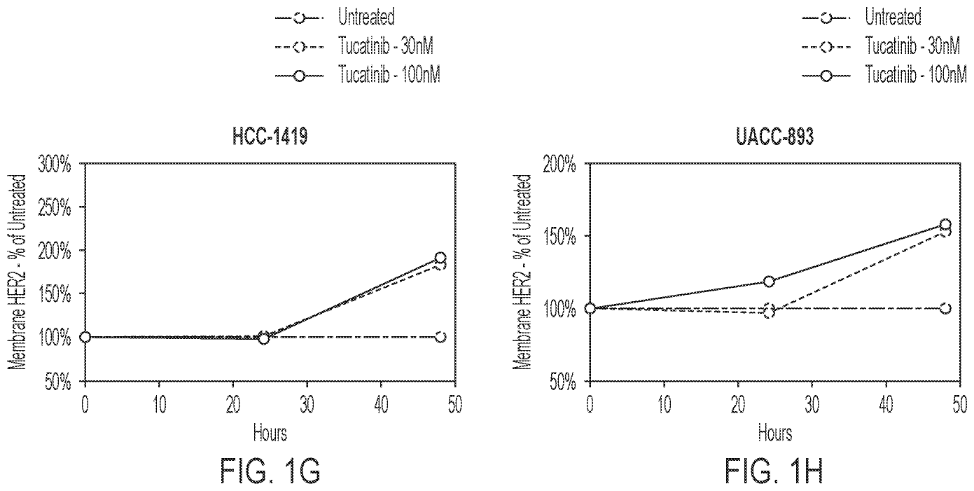

This study demonstrated the effect of either 30 nM or 100 nM tucatinib on total HER2 protein levels or plasma membrane-bound HER2 protein levels in four different HER2-amplified breast cancer cell lines (BT-474, SK-BR-3, HCC-1419, and UACC-893) after treatment with tucatinib at either dose for the duration of 24 hours and 48 hours. Protein lysates were generated from cells harvested at each time point. HER2 total protein levels were determined by western blot analysis using a WES system, and normalized against GAPDH levels as a loading control. As shown in FIG. 1A-1D, in all four cell lines tested, HER2 total protein levels increased after treatment with tucatinib relative to untreated cell lines. Total HER2 protein levels are shown as a percentage of the total HER2 protein levels of untreated cells. Plasma membrane-bound levels of HER2 were determined by quantitative FACS (qFACS) analysis after exclusion of dead cells. As shown in FIG. 1E-1H, treatment with either 30 nM or 100 nM increased plasma-membrane-bound HER2 protein levels in all four cell lines relative to untreated cell lines. Plasma membrane-bound HER2 protein levels are shown as a percentage of the plasma membrane-bound HER2 protein levels of untreated cells.

### Example 3: Treatment with Tucatinib Increased Dwell Time of HER2 at the Cell Surface and was Followed by Rapid Internalization and Lysosomal Processing

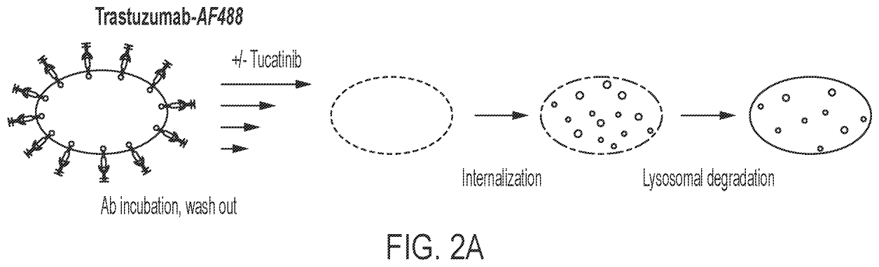

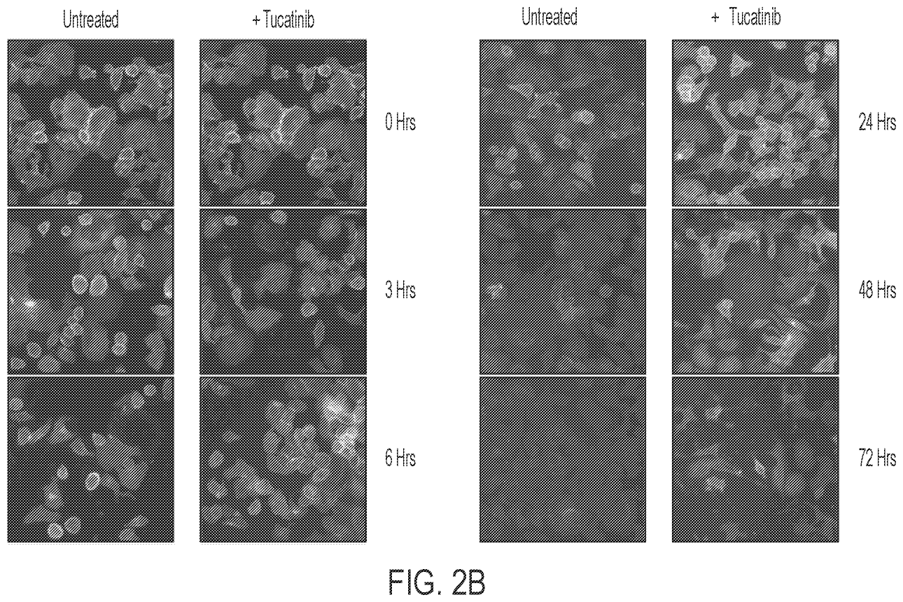

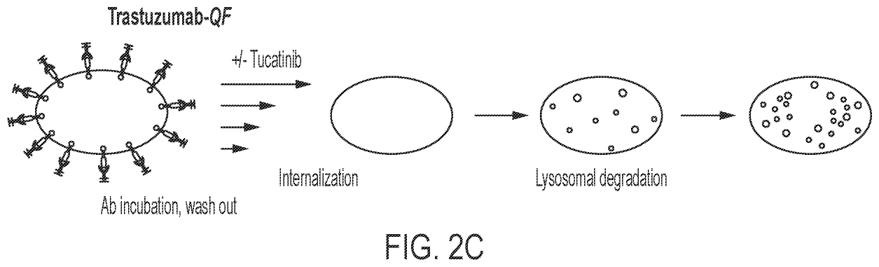

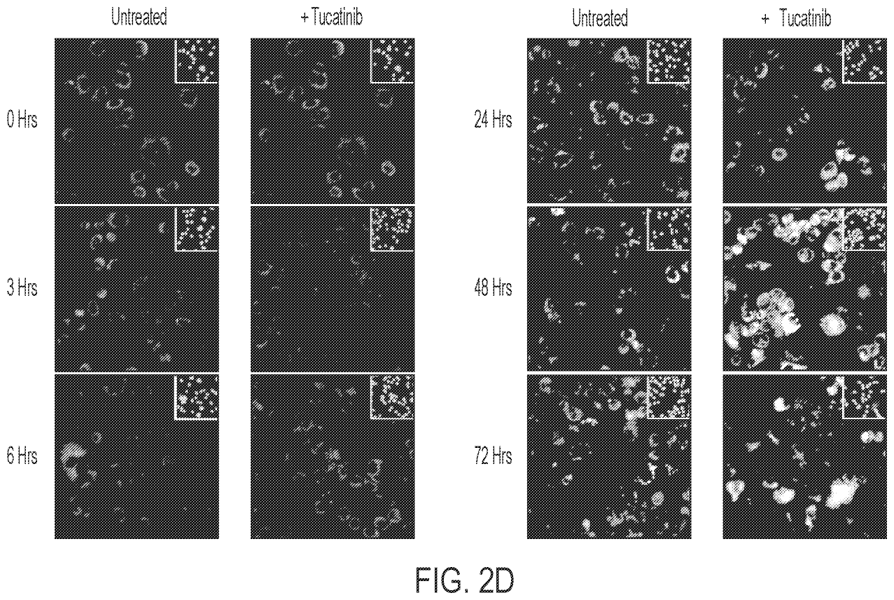

To probe the dynamics of HER2 at the cell surface upon binding to antibody therapeutics in the presence or absence of tucatinib (100 nM), SK-BR-3 cells were incubated with fluorescently-labeled trastuzumab (Trastuzumab-AF488) to mark HER2 at the cell surface. Excess antibody was washed out. Cells were imaged at time points spanning 72 hours to observe internalization of surface-bound antibody. FIG. 2A shows a schematic of a HER2 internalization assay using Trastuzumab-AF488. FIG. 2B shows the results of a HER2 internalization assay using Trastuzumab-AF488. Concurrent experiments were conducted with trastuzumab labeled with QF (Trastuzumab-QF) in the presence of chloroquine, a quenched fluorophore which fluoresces upon lysosomal processing and can serve as a proxy for antibody catabolism. FIG. 2C shows a schematic of a HER2 internalization assay using Trastuzumab-QF. FIG. 2D shows the results of a HER2 internalization assay using Trastuzumab-QF. These experiments demonstrate that treatment with tucatinib increased dwell time of HER2 at the cell surface (FIG. 2B) and was followed by rapid internalization and lysosomal processing (FIG. 2D).

Without wishing to be bound by any theory, the increase in overall and cell-membrane localized HER2 levels in response to tucatinib as shown in Example 2 and the initial increase in dwell time of HER2 at the cell surface in response to tucatinib as shown in the current example may provide a mechanistic rationale for why the co-administration of tucatinib with an anti-HER2 antibody can have synergistic effects. The administration of tucatinib may mediate increased receptor binding of anti-HER2 antibody therapeutics.

### Example 4: A Randomized, Double-Blind, Phase 3 Study of Tucatinib or Placebo in Combination with Trastuzumab and Pertuzumab as Maintenance Therapy for Metastatic HER2+ Breast Cancer

Described here is a randomized, double-blind, placebo-controlled, international, multicenter, phase 3 study designed to evaluate the efficacy and safety of tucatinib in combination with trastuzumab and pertuzumab as maintenance therapy in subjects with advanced HER2+ breast cancer, including subjects who have had prior treatment with a taxane, trastuzumab, and pertuzumab. Subjects will be randomized in a 1:1 ratio to receive 21-day cycles of either tucatinib in combination with trastuzumab and pertuzumab or placebo in combination with trastuzumab and pertuzumab. Randomization will be stratified by diagnosis (de novo versus recurrent), hormone receptor status (positive versus negative), and presence or history of brain metastases (yes versus no).

The study will evaluate the efficacy, safety, and pharmacokinetics (PK) of tucatinib in combination with trastuzumab and pertuzumab as maintenance therapy in subjects with advanced HER2+ breast cancer. Specific objectives and corresponding endpoints for the study are summarized in the table, below.

| Primary Objective | Corresponding Primary Endpoint |
|---|---|
| Compare PFS by investigator per Response | PFS, defined as the time from randomization to |
| Evaluation Criteria in Solid Tumors | investigator-assessed documented disease |
| (RECIST) v1.1 between treatment | progression per RECIST v1.1, or death from any |
| arms | cause, whichever occurs first |
| Key Secondary Objective | Corresponding Key Secondary Endpoint |
| Compare OS between treatment arms | OS, defined as the time from randomization to death |
| | from any cause |
| Other Secondary Objectives | Corresponding Secondary Endpoints |
| Evaluate PFS by blinded independent central | PFS, defined as the time from randomization to |
| review (BICR) per RECIST v1.1 | documented disease progression (as determined |
| | by BICR per RECIST v1.1), or death from any |
| | cause, whichever occurs first |
| Assess the change in health-related quality | Time to deterioration of HRQoL, defined as time to |
| of life (HRQoL) | 10-point decrease in the global health status/QoL |
| | scale of the European Organization for Research |
| | and Treatment of Cancer (EORTC) quality of |
| | life questionnaire (QLQ-C30) |
| Evaluate PFS in the brain | CNS-PFS, defined as the time from randomization to |
| | investigator-assessed disease progression in |
| | brain (RECIST v1.1), or death from any cause, |
| | whichever occurs first |
| Evaluate the safety and tolerability of | AEs |
| tucatinib in combination with | Clinical laboratory assessments |
| trastuzumab and pertuzumab | Incidence of dose holding, dose reductions, and |
| | discontinuations of tucatinib |
| | Incidence of dose holding and discontinuations of |
| | trastuzumab |
| | Incidence of dose holding and discontinuations of |
| | pertuzumab |
| Evaluate the PK of tucatinib | Plasma concentrations of tucatinib |
| Exploratory Objectives | Corresponding Exploratory Endpoints |
| Explore correlations between tissue/cell free | Potential biomarkers of response and/or resistance, |
| circulating tumor (ct)DNA biomarkers | from tumor samples and cell free ctDNA |
| and clinical outcomes | |
| Evaluate health utilities | HRQoL utilities as assessed with the European Quality |
| | of Life 5 Dimension 5 Level (EQ-5D-5L) |
| | instrument |
| | Change from baseline in global health status/QoL and |
| | physical functional scales of the EORTC |
| | QLQ-C30 |

Subjects will be randomized in a 1:1 ratio to receive 21-day cycles of treatment in 1 of the following 2 treatment groups: (1) Control arm: Placebo given PO BID plus trastuzumab and pertuzumab every 21 days; (2) Experimental arm: Tucatinib 300 mg PO BID plus trastuzumab and pertuzumab every 21 days.

Trastuzumab and pertuzumab will be administered as follows: trastuzumab will be given intravenously (IV) at a dose of 6 mg/kg or subcutaneously (SC) at a fixed dose of 600 mg, once every 21 days AND pertuzumab will be given IV at 420 mg every 21 days. In the alternative, a fixed dose combination of 600 mg pertuzumab, 600 mg trastuzumab, and 20,000 units hyaluronidase will be given SC, once every 21 days, in lieu of trastuzumab and pertuzumab individually. For IV trastuzumab, if dosing of trastuzumab has been held for >4 weeks, an IV loading dose of 8 mg/kg will be given. For IV pertuzumab, if dosing of pertuzumab has been held for at least 6 weeks, an IV loading dose of 840 mg will be given.

Tucatinib or placebo will be dispensed to subjects in a double-blinded manner. Cycles will be planned to be 21 days in length, with dispensation of tucatinib or placebo planned for Day 1 of each cycle. Study treatment will continue until unacceptable toxicity, disease progression (ideally verified radiologically per RECIST v1.1), withdrawal of consent, or study closure. No crossover from placebo to tucatinib will be allowed. Tucatinib and placebo will be administered by PO route; 300 mg will be administered PO BID from Cycle 1, Day 1 onwards. Tucatinib or placebo will be taken once in the morning and once in the evening (PO BID), with approximately 8 to 12 hours between doses in the same calendar day. Dose modifications of tucatinib or placebo may be prescribed. Up to 3 dose reductions of tucatinib or placebo are allowed, but dose reductions to below 150 mg BID are not allowed. Subjects who, in the opinion of the investigator, would require a dose reduction to less than 150 mg BID, or who would require a potential fourth dose reduction of tucatinib, should discontinue. The recommended dose reductions are 250 mg PO BID, 200 mg PO BID, and finally 150 mg PO BID. If a scheduled dose is missed, and less than 6 hours have passed since the scheduled dosing time, subjects will be recommended to immediately take the dose. If more than 6 hours have passed, subjects will be recommended to skip the dose and wait for the next regularly-scheduled dose. Tucatinib or placebo may be taken with or without food.

Dose reductions of tucatinib or placebo will be allowed. Dose holding or discontinuation of tucatinib or placebo, trastuzumab, and/or pertuzumab will also be allowed as needed for subject safety. In the absence of progression, subjects who discontinue trastuzumab and pertuzumab due to drug related toxicity may continue receiving tucatinib/placebo alone, and subjects who discontinue tucatinib/placebo may continue to receive trastuzumab and pertuzumab. If a subject discontinues trastuzumab or pertuzumab, they are required to discontinue both. Safety will be monitored in an ongoing, blinded basis throughout the study.

Disease response per RECIST v1.1 will be assessed. Treatment decisions will be made based upon local radiologic assessment. Response assessments for each subject will continue until disease progression per RECIST v1.1 has been documented, or end of treatment (EOT), whichever is later. Survival and other follow up assessments will continue until study closure or withdrawal of consent.

Blood samples for PK assessment of trough tucatinib drug levels will be collected in all subjects on Day 1 of Cycles 2 to 6, prior to administration of tucatinib. On Day 1 of Cycle 3, blood samples will also be collected post dose for PK assessments of peak levels of tucatinib 1 to 4 hours after administration of tucatinib. Plasma concentrations of tucatinib will be determined using validated liquid chromatography (LC) mass spectrometry (MS)/MS methods. PK parameters will be summarized using descriptive statistics. Health-related QoL will be assessed at protocol-specified time points using standardized assessment tools including the EQ 5D 5L instrument and the EORTC QLQ C30.

The study population will comprise subjects who must meet all of the enrollment criteria to be eligible. The inclusion criteria are:
1. Have centrally confirmed HER2+ breast carcinoma per 2018 American Society of Clinical Oncologists (ASCO)-College of American Pathologists (CAP) guidelines.
   a. Tissue blocks or slides must be submitted and confirmed as HER2+ by a sponsor-designated central laboratory prior to randomization.
2. Have unresectable locally advanced or metastatic (hereafter referred to as “advanced”) disease; if recurrent (after [neo]adjuvant therapy), there must be a minimum 6-month treatment-free interval from any trastuzumab or pertuzumab received in the early breast cancer setting to the diagnosis of advanced HER2+ disease. Prior standard of care therapy for early breast cancer is permitted (eg, prior T-DM1); however, Exclusion Criterion 1 should be noted.
3. Have received 4-8 cycles (21-day cycles) of previous treatment with trastuzumab, pertuzumab, and taxane as first-line therapy for advanced HER2+ breast cancer with no evidence of disease progression (per investigator judgement)
   a. Subjects receiving <6 cycles (ie, 4-5 cycles) of taxane are only eligible if the taxane was stopped early due to intolerable toxicity (eg, documented neuropathy impacting function).
   b. Subjects are permitted to receive trastuzumab and pertuzumab for 1 additional cycle (after completion of chemotherapy) during screening to allow completion of screening procedures. Study treatment should begin within 6 weeks (±3 days) from the start of the last cycle of trastuzumab, pertuzumab, and chemotherapy.
   c. Subjects newly found to have asymptomatic brain metastasis during screening and treated with local CNS-directed therapy are permitted to receive trastuzumab and pertuzumab for up to 2 additional cycles (after completion of chemotherapy) to allow for the mandatory washout period (see Inclusion Criterion 15 and Exclusion Criterion 14). Study treatment should begin within 9 weeks (±3 days) from the start of the last cycle of trastuzumab, pertuzumab, and chemotherapy.
4. Known hormone receptor status (per local guidelines; may be hormone receptor positive [HR+] or negative [HR−])
5. Be at least 18 years of age, and legally an adult at time of consent
6. Have Eastern Cooperative Oncology Group Performance Status (ECOG PS) of 0 or 1
7. Have adequate hepatic function as defined by the following:
   a. Total bilirubin ≤1.5× upper limit of normal (ULN), except for subjects with known Gilbert's disease who may enroll if the conjugated bilirubin is ≤1.5×ULN
   b. Transaminases (AST/serum glutamic oxaloacetic transaminase [SGOT] and ALT/serum glutamic pyruvic transaminase [SGPT])≤3×ULN (≤5×ULN if liver metastases are present)
8. Have adequate baseline hematologic parameters as defined by:
   a. Absolute neutrophil count (ANC) ≥1×10³/μL
   b. Platelet count ≥100×10³/μL
   c. Hemoglobin ≥9 g/dL
   d. In subjects receiving any transfusion before study entry, the above hematologic parameters must be met in absence of any transfusion for ≥14 days prior.
   e. In subjects receiving growth factors before study entry, the above hematologic parameters must be met in absence of relevant growth factor use for 14 days prior.
9. Have a serum or plasma creatinine ≤1.5× institutional ULN.
10. Have left ventricular ejection fraction (LVEF) ≥50% as assessed by echocardiogram (ECHO) or multiple-gated acquisition scan (MUGA) documented within 4 weeks prior to first dose of study treatment.
11. For subjects of childbearing potential, the following stipulations apply:
   a. Must have a negative serum or urine pregnancy test (minimum sensitivity of 25 mIU/mL or equivalent units of beta human chorionic gonadotropin [β-hCG]) result within 7 days prior to the first dose of study treatment. A subject with a false positive result and documented verification that the subject is not pregnant is eligible for participation.
   b. Must agree not to try to become pregnant during the study and for at least 7 months after the final dose of study drug
   c. Must agree not to breastfeed or donate ova, starting at time of informed consent and continuing through at least 7 months after the final dose of study drug
   d. If sexually active in a way that could lead to pregnancy, must consistently use 2 highly effective methods of birth control, starting at the time of informed consent and continuing throughout the study and for at least 7 months after the final dose of study drug
12. For male subjects, the following stipulations apply:
   a. Must agree not to donate sperm starting at the time of informed consent and continuing throughout the study period and for at least 7 months after the final dose of study drug
   b. If able to father children and sexually active with a person of childbearing potential in a way that could lead to pregnancy, must consistently use 2 highly effective methods of birth control, starting at time of informed consent and continuing throughout the study and for at least 7 months after the final dose of study drug
   c. Male subjects who are sexually active with a person who is pregnant or breastfeeding, must consistently use a male condom, starting at time of informed consent and continuing throughout the study and for at least 7 months after the final dose of study drug
13. Provide signed informed consent per a consent document that has been approved by an institutional review board or independent ethics committee (IRB/IEC) prior to initiation of any study-related tests or procedures that are not part of standard-of-care for the subject's disease
14. Be willing and able to comply with study procedures
15. CNS Inclusion—Based on screening contrast brain magnetic resonance imaging (MRI), subjects may have any of the following:
   a. No evidence of brain metastases
   b. Untreated brain metastases which are asymptomatic and, if identified on prior brain imaging, without evidence of progression since starting first-line induction therapy with trastuzumab, pertuzumab, and taxane
   c. Previously treated brain metastases which are asymptomatic
      i. Brain metastases previously treated with local therapy must not have progressed since treatment
         a. Time since whole brain radiation therapy (WBRT) is ≥14 days prior to first dose of study treatment, time since stereotactic radiosurgery (SRS) is ≥7 days prior to first dose of study treatment, or time since surgical resection is ≥28 days prior to first dose of study treatment
      ii. Relevant records of any CNS treatment must be available to allow for classification of target and non-target lesions:

The exclusion criteria include:
1. Have previously been treated with any anti-HER2 and/or anti-epidermal growth factor receptor (EGFR) tyrosine kinase inhibitor including pyrotinib, lapatinib, tucatinib, neratinib, and afatinib (except neratinib if given in extended adjuvant setting and at least 12 months have elapsed from the last neratinib dose to the start of study drug) or are currently participating in another interventional clinical trial
2. Unable for any reason to undergo contrast MRI of the brain
3. History of allergic reactions to trastuzumab, pertuzumab, or compounds chemically or biologically similar to tucatinib, except for Grade 1 or 2 infusion-related reactions (IRRs) to trastuzumab that were successfully managed, known allergy to one of the excipients in the study drugs, or hypersensitivity to murine proteins
4. Are positive for Hepatitis B by surface antigen expression, positive for Hepatitis C infection, or the presence of known chronic liver disease. Subjects who have been treated for Hepatitis C infection are permitted if they have documented sustained virologic response of at least 12 weeks. The latest local guidelines should be followed regarding the testing of Hepatitis B DNA levels by polymerase chain reaction (PCR). Subjects with Hepatitis B DNA levels by PCR that require nucleoside analogue or other therapies are not eligible for the trial.
5. Subjects known to be positive for human immunodeficiency virus (HIV) are excluded if they meet any of the following criteria:
   a. CD4+ T-cell count of <350 cells/μL
   b. Detectable HIV viral load
   c. History of an opportunistic infection within the past 12 months
   d. On stable antiretroviral therapy for <4 weeks
6. Are pregnant, breastfeeding, or planning a pregnancy from time of informed consent until
7 months after the final dose of study drug
7. Have inability to swallow pills or have significant GI disease or surgery which would preclude the adequate oral absorption of medications
8. Have used a strong CYP2C8 inhibitor within 5 half-lives of the inhibitor, or have used a strong CYP3A4 or CYP2C8 inducer within 5 days prior to first dose of study treatment.
9. Have current conditions of symptomatic congestive heart failure, unstable angina pectoris, uncontrolled hypertension, or cardiac arrhythmia or history of myocardial infarction within 6 months prior to randomization
10. Have any other medical, social, or psychosocial factors that, in the opinion of the investigator, could impact safety or compliance with study procedures
11. Have evidence within 2 years of the start of study treatment of another malignancy that required systemic treatment
12. Have ongoing ≥Grade 2 toxicity from first-line induction therapy (ie, trastuzumab, pertuzumab, and taxane) with the exceptions of alopecia, neuropathy, and nail toxicity
13. Have ongoing ≥Grade 2 diarrhea
14. CNS Exclusion—Based on screening brain MRI and clinical assessment, subjects must not have any of the following:
   a. Symptomatic brain metastasis
   b. Progression of brain metastases since starting first-line trastuzumab, pertuzumab, and taxane
   c. Ongoing use of systemic corticosteroids at a total daily dose of >2 mg of dexamethasone (or equivalent). For subjects requiring systemic steroids for control of comorbidities (eg, asthma or autoimmune diseases), daily dose must not exceed 2 mg dexamethasone (or equivalent).
   d. Any untreated brain lesion in an anatomic site which may pose risk to subject (eg, brain stem lesions). Subjects who successfully undergo local treatment for such lesions may be permitted to rescreen, if otherwise eligible, after discussion with, and approval by, the medical monitor.
   e. Known or suspected leptomeningeal disease (LMD) as documented by the investigator
   f. Poorly controlled (>1/week) seizures, or other persistent neurologic symptoms despite CNS-directed therapy for brain metastasis

A subject's study treatment may be discontinued for any of the following reasons:
Progressive disease (CNS-only progressive disease is allowed)
Adverse event (AE)
Pregnancy
Investigator decision, non-AE
Subject decision, non-AE
Study termination by sponsor
Other, non-AE
   In the absence of progression:
subjects who discontinue trastuzumab and pertuzumab due to drug-related toxicity may continue receiving tucatinib/placebo alone.
subjects who discontinue tucatinib/placebo may continue to receive trastuzumab and pertuzumab.
if a subject discontinues trastuzumab or pertuzumab, they are required to discontinue both.

Blood samples for PK assessment of trough tucatinib drug levels will be collected in all subjects on Day 1 of Cycles 2 to 6 (prior to administration of tucatinib) and at EOT. On Day 1 of cycle 3, PK assessments of peak levels of tucatinib will be performed 1 to 4 hours after administration of tucatinib. Plasma concentrations of tucatinib will be determined using validated LC-MS/MS methods.

Relationships of biomarker parameters (e.g., baseline values, absolute and relative changes from baseline) to efficacy, safety and PK parameters may be explored in peripheral blood and tumor tissue. Exploratory, predictive, and prognostic biomarkers associated with response and/or resistance observations may be monitored before and after treatment with tucatinib. Correlative studies may be conducted to gain a better understanding of target-response relationship, predictive/prognostic biomarkers, MOA, resistance mechanisms, and pharmacodynamics.

Subjects will have HER2 status determined by immunohistochemistry (IHC) and fluorescence in situ hybridization (FISH) in samples prior to enrollment. Biomarker assessments may include an exploratory assessment of mutations as potential biomarkers of response. Additional analyses including but not limited to IHC or next generation sequencing (NGS) may be performed to interrogate biomarkers that are associated with tumor growth, survival, and resistance to targeted therapeutics. This assessment may enable the correlation of additional biomarkers with treatment outcome and may ultimately guide or refine subject selection strategies to better match tucatinib regimens with tumor phenotype/genotype in the future.

Exploratory biomarkers of clinical activity may be assessed in blood samples collected at screening and at EOT. Retrospective exploratory analysis may be conducted (in subjects who consented to genetic analysis) including sequencing of cell free ctDNA to investigate possible associations with mechanism of resistance to treatments and dynamic changes associated with study treatment.

Response will be assessed according to RECIST v1.1, which are standardized criteria for evaluating response in solid tumors. The schedule for tumor balances measurement of tumor control (and thus appropriateness of continuing study treatment) with the burden of imaging. The safety measures that will be used in this trial are considered standard procedures for evaluating the potential adverse effects of study medications. Additional safety assessments may be performed as clinically appropriate. AEs and clinical laboratory data will be graded using standardized criteria for oncology (NCI CTCAE version 5.0). Pharmacokinetic assessments are common in clinical studies to help characterize dose exposure response relationships. The EQ-5D-5L is a validated instrument for use as a measure of HRQoL. This PRO has been incorporated into previous clinical trials that seek to quantify the HRQoL in subjects. The QLQ-C30 (Version 3.0) is a validated questionnaire developed by the EORTC to assess the QoL of cancer subjects. These PROs have been incorporated into previous clinical trials that seek to quantify the QoL in subjects.

What is claimed is:

1. A method for treating a solid tumor in a subject comprising administering a combination of tucatinib, or salt or solvate thereof, and at least one anti-HER2 antibody to the subject, wherein the solid tumor has one or more HER2 alterations.

2. The method of claim 1, wherein the subject exhibits progression free survival (PFS) of at least 1 month after administration of the tucatinib, or salt or solvate thereof, and the at least one anti-HER2 antibody.

3. The method of claim 1 or claim 2, wherein the subject exhibits an overall survival (OS) of at least 2 months after administration of the tucatinib, or salt or solvate thereof, and the at least one anti-HER2 antibody.

4. The method of any one of claims 1-3, wherein the subject exhibits a greater than 10% reduction in the risk of disease progression as compared to a subject administered the at least one anti-HER2 antibody alone.

5. The method of claim 4, wherein the subject exhibits a greater than 25% reduction in the risk of disease progression as compared to a subject administered the at least one anti-HER2 antibody alone.

6. The method of claim 4, wherein the subject exhibits a greater than 30% reduction in the risk of disease progression as compared to a subject administered the at least one anti-HER2 antibody alone.

7. The method of any one of claims 1-6, wherein the subject exhibits a greater than 10% reduction in the risk of death as compared to a subject administered the at least one anti-HER2 antibody alone.

8. The method of claim 7, wherein the subject exhibits a greater than 25% reduction in the risk of death as compared to a subject administered the at least one anti-HER2 antibody alone.

9. The method of claim 7, wherein the subject exhibits a greater than 30% reduction in the risk of death as compared to a subject administered the at least one anti-HER2 antibody alone.

10. The method of any one of claims 1-9, wherein following administration of the tucatinib, or salt or solvate thereof, and the at least one anti-HER2 antibody, for nine months the subject has an estimated PFS rate of greater than 20%.

11. The method of claim 10, wherein the subject has an estimated PFS rate of greater than 30%.

12. The method of claim 10, wherein the subject has an estimated PFS rate of greater than 40%.

13. The method of claim 10, wherein the subject has an estimated PFS rate of greater than 45%.

14. The method of any one of claims 1-9, wherein following administration of the tucatinib, or salt or solvate thereof, and the at least one anti-HER2 antibody, for twelve months the subject has an estimated PFS rate of greater than 15%.

15. The method of claim 14, wherein the subject has an estimated PFS rate of greater than 20%.

16. The method of claim 14, wherein the subject has an estimated PFS rate of greater than 30%.

17. The method of claim 14, wherein the subject has an estimated PFS rate of greater than 40%.

18. The method of any one of claims 1-9, wherein following administration of the tucatinib, or salt or solvate thereof, and the at least one anti-HER2 antibody, for fifteen months the subject has an estimated PFS rate of greater than 15%.

19. The method of claim 18, wherein the subject has an estimated PFS rate of greater than 20%.

20. The method of claim 18, wherein the subject has an estimated PFS rate of greater than 25%.

21. The method of claim 18, wherein the subject has an estimated PFS rate of greater than 30%.

22. The method of any one of claims 1-21, wherein following administration of the tucatinib, or salt or solvate thereof, and the at least one anti-HER2 antibody, for twenty-four months the subject has an estimated OS rate of greater than 25%.

23. The method of claim 22, wherein the subject has an estimated OS rate of greater than 35%.

24. The method of claim 22, wherein the subject has an estimated OS rate of greater than 40%.

25. The method of any one of claims 1-21, wherein following administration of the tucatinib, or salt or solvate thereof, and the at least one anti-HER2 antibody, for thirty months the subject has an estimated OS rate of greater than 20%.

26. The method of claim 25, wherein the subject has an estimated OS rate of greater than 25%.

27. The method of claim 25, wherein the subject has an estimated OS rate of greater than 30%.

28. The method of anyone of claims 1-27, wherein the tucatinib, or salt or solvate thereof, and the at least one anti-HER2 antibody are administered to the subject on a 21-day treatment cycle.

29. The method of claim 28, wherein the at least one anti-HER2 antibody is administered to the subject on day 1 of the 21-day treatment cycle.

30. The method of any one of claims 1-29, wherein the at least one anti-HER2 antibody is administered once about every 3 weeks.

31. The method of any one of claims 1-30, wherein the tucatinib, or salt or solvate thereof, is administered to the subject twice per day.

32. The method of any one of claims 1-31, wherein the one or more HER2 alterations is a HER2 mutation, wherein the HER2 mutation comprises at least one amino acid substitution, insertion, or deletion compared to the amino acid sequence of SEQ ID NO:1.

33. The method of claim 32, wherein the HER2 mutation is an activating mutation.

34. The method of claim 32 or claim 33, wherein the HER2 mutation is a mutation in the extracellular domain, the kinase domain, or the transmembrane/juxtamembrane domain, or any combination thereof.

35. The method of claim 34, wherein the HER2 mutation is a mutation in the extracellular domain selected from the group consisting of G309A, G309E, S310F, S310Y, C311R, C311S, and C334S.

36. The method of claim 34, wherein the HER2 mutation is a mutation in the kinase domain at an amino acid residue selected from the group consisting of Y772, G776, G778, and T798.

37. The method of claim 34, wherein the HER2 mutation is a G776 YVMA insertion.

38. The method of claim 34, wherein the HER2 mutation is a mutation in the kinase domain selected from the group consisting of T733I, L755P, L755S, 1767M, L768S, D769N, D769Y, D769H, V777L, V777M, L841V, V8421, N857S, T862A, L869R, H878Y, and R896C.

39. The method of claim 34, wherein the HER2 mutation is a mutation in the kinase domain at an amino acid residue V697.

40. The method of claim 34, wherein the HER2 mutation is a mutation in the transmembrane/juxtamembrane domain selected from the group consisting of S653C, I655V, V659E, G660D, and R678Q.

41. The method of any one of claims 32-40, wherein the HER2 mutation is determined by using next generation sequencing (NGS).

42. The method of any one of claims 1-31, wherein the one or more HER2 alterations is HER2 overexpression/amplification.

43. The method of claim 42, wherein the HER2 overexpression is 3+ overexpression as determined by immunohistochemistry (IHC).

44. The method of claim 42, wherein the HER2 amplification is determined by an in situ hybridization assay.

45. The method of claim 44, wherein the in situ hybridization assay is fluorescence in situ hybridization (FISH) assay.

46. The method of claim 44, wherein the in situ hybridization assay is chromogenic in situ hybridization.

47. The method of claim 42, wherein the HER2 amplification is determined in tissue by NGS.

48. The method of claim 42, wherein the HER2 amplification is determined in circulating tumor DNA (ctDNA) by a blood-based NGS assay.

49. The method of any one of claims 1-48, wherein the solid tumor is a HER2+ solid tumor.

50. The method of any one of claims 1-49, wherein the solid tumor is a metastatic solid tumor.

51. The method of any one of claims 1-50, wherein the solid tumor is locally-advanced.

52. The method of any one of claims 1-51, wherein the solid tumor is unresetable.

53. The method of any one of claims 1-52, wherein the solid tumor is selected from the group consisting of cervical cancer, uterine cancer, gallbladder cancer, cholangiocarcinoma, urothelial cancer, lung cancer, breast cancer, gastroesophageal cancer, and colorectal cancer.

54. The method of claim 53, wherein the solid tumor is gallbladder cancer and the subject has completed at least one prior line of treatment for the gallbladder cancer.

55. The method of claim 54, wherein the prior line of treatment is selected from the group consisting of chemotherapy, endocrine therapy, and targeted therapy.

56. The method of claim 53, wherein the solid tumor is cholangiocarcinoma and the subject has completed at least one prior line of treatment for the cholangiocarcinoma.

57. The method of claim 56, wherein the prior line of treatment is selected from the group consisting of chemotherapy, endocrine therapy, and targeted therapy.

58. The method of claim 53, wherein the lung cancer is non-small cell lung cancer (NSCLC).

59. The method of claim 58, wherein the NSCLC is non-squamous NSCLC.

60. The method of claim 59, wherein the subject has relapsed from standard of care treatment.

61. The method of claim 59, wherein the subject is refractory to standard of care treatment.

62. The method of claim 59, wherein no standard of care treatment is available for the subject.

63. The method of claim 53, wherein the solid tumor is breast cancer.

64. The method of claim 63, wherein the subject has completed at least one prior line of treatment for the breast cancer.

65. The method of claim 64, wherein the prior line of treatment is selected from the group consisting of chemotherapy, endocrine therapy, and targeted therapy.

66. The method of any one of claims 63-65, wherein the breast cancer is hormone receptor positive (HR+) breast cancer.

67. The method of any one of claims 63-66, wherein the breast cancer is a metastasized breast cancer.

68. The method of claim 66 or claim 67, wherein the subject is administered fulvestrant in combination with the tucatinib, or salt or solvate thereof, and the at least one anti-HER2 antibody.

69. The method of claim 68, wherein the fluvestrant is administered at a dose of 500 mg.

70. The method of claim 68 or claim 69, wherein the route of administration of the fluvestrant is administered intramuscular (IM).

71. The method of any one of claims 68-70, wherein the fluvestrant is administered on day 1 of the first 21-day treatment cycle.

72. The method of claim 71, wherein the fluvestrant is administered once about every 4 weeks.

73. The method of claim 71 or claim 72, wherein the fluvestrant is further administered on day 15 of the first 21-day treatment cycle.

74. The method of any one of claims 1-73, wherein the tucatinib, or salt or solvate thereof, is administered to the subject at a dose of about 150 mg to about 650 mg.

75. The method of claim 74, wherein the tucatinib, or salt or solvate thereof, is administered to the subject at a dose of about 300 mg.

76. The method of any one of claims 1-75, wherein the tucatinib, or salt or solvate thereof, is administered to the subject orally.

77. The method of any one of claims 1-76, wherein the at least one anti-HER2 antibody is administered to the subject at a dose of about 4 mg/kg to about 10 mg/kg.

78. The method of claim 77, wherein the at least one anti-HER2 antibody is administered to the subject at a dose of about 6 mg/kg of the subject's body weight.

79. The method of claim 78, wherein the at least one anti-HER2 antibody is administered to the subject at a dose of about 8 mg/kg of the subject's body weight.

80. The method of claim 77, wherein the at least one anti-HER2 antibody is administered to the subject at a dose of about an initial dose of about 8 mg/kg followed by subsequent doses of about 6 mg/kg.

81. The method of claim 77, wherein the dose of the at least one anti-HER2 antibody administered during the first 21-day treatment cycle is 8 mg/kg of the subject's body weight and the dose administered during the subsequent 21-day treatment cycles is 6 mg/kg of the subject's bodyweight.

82. The method of any one of claims 1-81, wherein the at least one anti-HER2 antibody is administered intravenously.

83. The method of any one of claims 1-82, wherein the at least one anti-HER2 antibody comprises one anti-HER2 antibody.

84. The method of any one of claims 1-83, wherein the at least one anti-HER2 antibody is trastuzumab, or a biosimilar thereof.

85. The method of any one of claims 1-83, wherein the at least one anti-HER2 antibody is trastuzumab.

86. The method of any one of claims 1-76, wherein the at least one anti-HER2 antibody comprises a first anti-HER2 antibody and a second anti-HER2 antibody.

87. The method of claim 86, wherein the first anti-HER2 antibody is administered to the subject at a dose of about 4 mg/kg to about 10 mg/kg.

88. The method of claim 87, wherein the first anti-HER2 antibody is administered to the subject at a dose of about 6 mg/kg of the subject's body weight.

89. The method of claim 86, wherein the first anti-HER2 antibody is administered at a dose of about 200 mg to about 1,000 mg.

90. The method of claim 89, wherein the first anti-HER2 antibody is administered at a dose of about 600 mg.

91. The method of any one of claims 86-90, wherein the second anti-HER2 antibody is administered to the subject at a dose of about 200 mg to about 1,000 mg.

92. The method of claim 91, wherein the second anti-HER2 antibody is administered at a dose of about 420 mg.

93. The method of claim 91, wherein the second anti-HER2 antibody is administered at a dose of about 600 mg.

94. The method of claim 86, wherein the first anti-HER2 antibody is administered at a dose of about 6 mg/kg and the second anti-HER2 antibody is administered at a dose of about 420 mg.

95. The method of any one of claims 86-94, wherein the first anti-HER2 antibody is administered intravenously.

96. The method of any one of claims 86-94, wherein the first anti-HER2 antibody is administered subcutaneously.

97. The method of any one of claims 86-96, wherein the second anti-HER2 antibody is administered intravenously.

98. The method of any one of claims 86-96, wherein the second anti-HER2 antibody is administered subcutaneously.

99. The method of claim 86, wherein the first anti-HER2 antibody is administered at a dose of 6 mg/kg intravenously or at a dose of about 600 mg subcutaneously; and wherein the second anti-HER2 antibody is administered at a dose of about 420 mg intravenously.

100. The method of claim 86, wherein the first anti-HER2 antibody and the second anti-HER2 antibody are administered in a pharmaceutical composition comprising about 600 mg of the first anti-HER2 antibody and 600 mg of the second anti-HER2 antibody: wherein the pharmaceutical composition is administered subcutaneously.

101. The method of claim 100, wherein the pharmaceutical composition further comprises hyaluronidase.

102. The method of claim 101, wherein the pharmaceutical composition comprises about 20,000 units hyaluronidase.

103. The method of any one of claims 86-102, wherein the first anti-HER2 antibody is trastuzumab, or a biosimilar thereof.

104. The method of any one of claims 86-103, wherein the second anti-HER2 antibody is pertuzumab, or a biosimilar thereof.

105. The method of any one of claims 86-104, wherein the first anti-HER2 antibody is administered about once every 3 weeks.

106. The method of any one of claims 86-105, wherein the second anti-HER2 antibody is administered about once every 3 weeks.

107. The method of any one of claims 1-106, wherein treating the subject results in a tumor growth inhibition (TGI) index of at least about 85%.

108. The method of any one of claims 1-107, wherein treating the subject results in a TGI index of about 100%.

109. The method of any one of claims 1-108, wherein one or more therapeutic effects in the subject is improved after administration of the tucatinib, or salt or solvate thereof, and the at least one anti-HER2 antibody to the subject relative to a baseline.

110. The method of claim 109, wherein the one or more therapeutic effects is selected from the group consisting of: size of a tumor derived from the solid tumor, objective response rate, duration of response, time to response, progression free survival and overall survival.

111. The method of any one of claims 1-110, wherein the size of a tumor derived from the solid tumor is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the solid tumor before administration of the tucatinib, or salt or solvate thereof, and the at least one anti-HER2 antibody.

112. The method of any one of claims 1-111, wherein the objective response rate is at least about 20%, at least about 25%, at least about 30%, at least about 35%, at least about 40%, at least about 45%, at least about 50%, at least about 60%, at least about 70%, or at least about 80%.

113. The method of any one of claims 1-112, wherein the subject exhibits progression-free survival of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody.

114. The method of any one of claims 1-113, wherein the subject exhibits overall survival of at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody.

115. The method of any one of claims 1-114, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the at least one anti-HER2 antibody.

116. The method of any one of claims 1-115, wherein the administration of the tucatinib, or salt or solvate thereof, increases the overall amount of HER2 in the solid tumor.

117. A method of increasing the overall amount of HER2 in a solid tumor comprising administering tucatinib, or salt or solvate thereof to a subject, wherein the administration of the tucatinib, or salt or solvate thereof, increases the overall amount of HER2 in the solid tumor.

118. The method of claim 116 or claim 117, wherein the overall amount of HER2 in the solid tumor is determined by Western blot analysis.

119. The method of any one of claims 1-118, wherein the administration of the tucatinib, or salt or solvate thereof, increases the amount of plasma membrane-bound HER2 in the solid tumor.

120. A method of increasing the amount of plasma membrane-bound HER2 in a solid tumor comprising administering tucatinib, or salt or solvate thereof to a subject, wherein the administration of the tucatinib, or salt or solvate thereof, increases the amount of plasma membrane-bound HER2 in the solid tumor.

121. The method of claim 119 or 120, wherein the amount of plasma membrane-bound HER2 in the solid tumor is determined by quantitative fluorescence activated cell sorting (qFACS).

122. The method of any one of claims 1-121, wherein the administration of the tucatinib, or salt or solvate thereof, increases the dwell time of HER2 at the cell surface.

123. A method of increasing dwell time of HER2 at the cell surface of a solid tumor comprising administering tucatinib, or salt or solvate thereof to a subject, wherein the administration of the tucatinib, or salt or solvate thereof, increases the dwell time of HER2 at the cell surface.

124. The method of any one of claims 1-123, wherein the administration of the tucatinib, or salt or solvate thereof, increases the internalization of plasma membrane-bound HER2.

125. A method of increasing internalization of plasma membrane-bound HER2 in a solid tumor comprising administering tucatinib, or salt or solvate thereof to a subject, wherein the administration of the tucatinib, or salt or solvate thereof, increases the internalization of plasma membrane-bound HER2.

126. The method of any one of claims 1-125, wherein the administration of the tucatinib, or salt or solvate thereof, increases the lysosomal degradation of HER2.

127. A method of increasing lysosomal degradation of HER2 in a solid tumor comprising administering tucatinib, or salt or solvate thereof to a subject, wherein the administration of the tucatinib, or salt or solvate thereof, increases the lysosomal degradation of HER2.

128. The method of any one of claims 1-127, wherein the subject is a human.

129. A kit comprising:
(a) tucatinib, or salt or solvate thereof;
(b) at least one anti-HER2 antibody; and
(c) instructions for using the kit in the method of any one of claims 1-125.

130. The kit of claim 129, wherein the at least one anti-HER2 antibody comprises trastuzumab.

131. The kit of claim 129 or claim 130, wherein the at least one anti-HER2 antibody comprises pertuzumab.
